(19) United States
(12) Patent Application Publication
(10) Pub. No.: US 2021/0145964 A1
(43) Pub. Date: May 20, 2021

(54) STROMA-TARGETING TREATMENT FOR PATIENTS WITH ELEVATED ADAM12 LEVELS

(71) Applicant: Academisch Medisch Centrum, Amsterdam Zuidoost (NL)

(72) Inventors: Maarten Fokke BIJLSMA, Amsterdam Zuidoost (NL); Hanneke Wilma Marlies VAN LAARHOVEN, Amsterdam Zuidoost (NL)

(21) Appl. No.: 16/685,100

(22) Filed: Nov. 15, 2019

(51) Int. Cl. A61K 39/395; A61K 31/4178; C07K 16/28; A61K 31/337; A61K 31/282; A61K 45/06; C12Q 1/37

(52) U.S. Cl. CPC A61K 39/3955; A61K 31/4178; C07K 16/2866; C07K 2317/24; A61K 31/282; A61K 45/06; C12Q 1/37; A61K 31/337

(57) ABSTRACT

The invention relates to a method for treating a human patient having a cancer characterized by at least one stroma-rich tumor, said human patient having increased disintegrin and metalloproteinase domain-containing protein 12 (ADAM12) levels in a bodily fluid, when compared to ADAM12 levels in a bodily fluid of a control person, the method comprising treating the human patient with a stroma-targeting agent. The invention further relates to a composition comprising a stroma-targeting agent for treating a human patient diagnosed with cancer characterized with at least one stroma-rich tumor, and one or more pharmaceutically acceptable excipients.

### FIELD OF THE INVENTION

The present invention relates to methods for treating a human patient having a cancer characterized by at least one stroma-rich tumor with a stroma-targeting agent.

### BACKGROUND OF THE INVENTION

Cancer is one of the leading death causes in the United States and the rest of the world. Adequate treatment of cancer is thus of utmost importance to increase cancer patients' survival. Esophageal cancer, for example, has a poor prognosis and currently ranks sixth in cancer related mortality [Pennathur et al., 2013. Lancet 381: 400-412]. A steep increase in the incidence of the esophageal adenocarcinoma (EAC) subtype is observed in Western countries. Patients eligible for curative treatment typically receive neoadjuvant chemo-radiation therapy, followed by surgery. The efficacy of this regimen is modest, and tumor cells often become resistant to the chemotherapeutics, indicating a need to identify the mechanisms that contribute to therapy resistance.

Research on therapy resistance has centered on tumor cell-intrinsic properties, but it is increasingly clear that the tumor microenvironment (TME) is important for this as well [Sun, 2015. Med Res Rev 35: 408-436]. Cancer associated fibroblasts (CAFs) comprise the majority of the TME and are suspected to exert tumor-promoting activities by their mechanical contributions to the stroma, as well as by secretion of cytokines [Kalluri and Zeisberg, 2006. Nat Rev Cancer 6: 392-401]. The presence of CAFs surrounding a tumor, as determined by expression of smooth muscle actin (α-SMA), is associated with poor survival in many solid malignancies, but the exact tumor-promoting activities of these cells may vary between cancer types. For example, the specific contributions of CAFs may depend on the cytokines that are produced by the fibroblasts.

Interleukin-6 (IL-6) is primarily known for its role in inflammation in response to a potentially harmful stimulus. IL6 expression may also be enhanced after exposure to anti-cancer drugs and ionizing radiation. IL-6 may also be expressed in the absence of therapeutic stress [Rodier et al., 2009. Nat Cell Biol 11: 973-979; Yun et al., 2012. Cancer Lett 323: 155-160]. In various cancer types, both tumor cells and CAFs can produce IL-6 [Gao et al., 2016. Oncol Rep 35: 3265-3274; Karakasheva et al., 2018. Cancer Res 78: 4957-4970; Wu et al., 2013. Radiat Oncol 8: 159; Xu et al., 2017. Exp Cell Res 351: 142-149]. The tumor-promoting activities of IL-6 are manifold and include the evasion of growth suppression by regulating the TP53 tumor suppressor gene [Hodge et al., 2005. Cancer Res 65: 4673-4682], mediating resistance against cell death [Leu et al., 2003. Oncogene 22: 7809-7818; Garcia-Tunon et al., 2005. Histopathology 47: 82-89], increasing stemness of tumor cells [Zhang et al., 2016. Cancer Sci 107: 746-754; Huang et al. 2016. Oncotarget 7: 62352-62363], and mediating tumor invasion and metastasis [Helbig et al., 2003. J Biol Chem 278: 21631-21638; Yadav et al., 2011. Mol Cancer Res 9: 1658-1667; Liu et al., 2015. Carcinogenesis 36: 459-468]. Also, stroma-derived IL-6 has been reported to be dysregulated in the metaplasia-dysplasia-EAC sequence of development of esophageal adenocarcinoma [Saadi et al., 2010. Proc Natl Acad Sci USA 107: 2177-2182].

There is thus a need for a better treatment method of human patients having a cancer characterized with at least one stroma-rich tumor.

### SUMMARY OF THE INVENTION

Human patients having cancer that is characterized by at least one stroma-rich tumor and having increased disintegrin and metalloproteinase domain-containing protein 12 (ADAM12) levels in a bodily fluid when compared to ADAM12 levels in a bodily fluid of a control person, are indicated to be susceptible to a stroma-targeting agent, such as an interleukin 6-targeting agent.

The invention therefore provides a method for treating a human patient having a cancer that is characterized by at least one stroma-rich tumor, said human patient having increased disintegrin and metalloproteinase domain-containing protein 12 (ADAM12) levels in a bodily fluid, when compared to ADAM12 levels in a bodily fluid of a human control. The method comprises treating the human patient with a stroma-targeting agent.

Said human patient may have at least one tumor size of more than 20 mm in diameter, have experienced a weight loss of more than 5%, have a lymph node ratio of more than 0.2, or a combination thereof.

The tumor size is preferably determined with a method comprising the steps of: (a) imaging the tumor with esophagogastroscopy, computed tomography, positron emission tomography, magnetic resonance imaging, or a combination thereof, and (b) determining a largest dimension of the tumor, wherein a length of said largest dimension is used as a proxy for the tumor size.

A lymph node ratio is preferably determined with a method comprising the steps of: (a) evaluating five or more lymph nodes for presence or absence of cancer cells; and (b) calculating the lymph node ratio by dividing an amount of lymph nodes with cancer cells by a total amount of evaluated lymph nodes.

Said human patient has preferably ADAM12 levels in the bodily fluid that is higher than 150 pg/mL, such as higher than 200 pg/mL, higher than 300 pg/mL. Said level of ADAM12 preferably is measured with an enzyme-linked immunosorbent assay (ELISA).

A preferred bodily fluid is blood, preferably blood serum.

A stroma-targeting agent preferably is a modulator of the renin-angiotensin system such as an angiotensin converting enzyme-inhibitor and/or an angiotensin II receptor blocker such as losartan, an interleukin 6 (IL-6)-targeting agent, preferably tocilizumab, siltuximab, olokizumab, elsilimomab, clazakizumab, sirukumab, sarilumab, vobarilizumab, or a combination thereof. Preferably, the stroma-targeting agent is tocilizumab, losartan, or a combination thereof.

In an embodiment, the stroma-targeting agent is administered orally, intravenously, subcutaneously, intramuscularly or a combination thereof, preferably intravenously by infusion, or subcutaneously.

A modulator of the renin-angiotensin system as a stroma-targeting agent is preferably administered in a dosage of between 0.1 mg and 2000 mg, Said modulator of the renin-angiotensin system is preferably administered once or twice daily.

An interleukin 6 (IL-6)-targeting agent as a stroma-targeting agent is preferably administered in a dosage of between 160 mg and 800 mg, Said stroma-targeting agent is preferably administered once every week or once every two weeks.

In an embodiment, treatment of said human patient additionally comprises surgery, chemotherapy, radiation therapy, targeted therapy, immunotherapy, hormone therapy, or a combination thereof.

In an embodiment, treating a human patient comprises administration of a stroma-targeting agent, combined with radiation therapy and/or chemotherapy and, optionally, surgery. Treating a human patient preferably comprises the steps of: a. radiation therapy, b. chemotherapy, c. administration of a stroma-targeting agent, and optionally d. surgery. Treating a human patient preferably comprises the steps of: a. radiation therapy, b. chemotherapy, c. administration of a stroma-targeting agent, and optionally d. surgery, in the indicated order.

In an embodiment, said patient has esophageal cancer, especially esophageal adenocarcinoma. For a patient having esophageal cancer, especially esophageal adenocarcinoma, a method of the invention may additionally comprise treating said patient with therapy including administration of trastuzumab, paclitaxel, cisplatin, a fluoropyrimidine such as 5-fluorouracil and capecitabine, oxaliplatin, irinotecan, carboplatin, ramucirumab, folinic acid (leucovorin), a combination of trifluridine with tipiracil (lonsurf), or a combination thereof.

Preferably, the patient is additionally treated with 5-fluorouracil, leucovorin, capecitabine, irinotecan, or a combination thereof such as 5-fluorouracil and leucovorin, or capecitabine and irinotecan.

The invention further provides a pharmaceutical composition comprising a stroma-targeting agent for treating a human patient diagnosed with cancer characterized with at least one stroma-rich tumor, and one or more acceptable excipients. Said stroma-targeting agent preferably is an interleukin 6 targeting agent. Said stroma-targeting agent preferably is an interleukin 6 (IL-6)-targeting agent, preferably selected from tocilizumab, siltuximab, olokizumab, elsilimomab, clazakizumab, sirukumab, sarilumab, vobarilizumab, or a combination thereof. Preferably, the stroma-targeting agent is tocilizumab.

The invention further provides a pharmaceutical composition, comprising a stroma targeting agent such as an interleukin 6 targeting agent and a chemotherapeutic agent. Said pharmaceutical composition may comprise a pharmaceutical composition comprising a stroma targeting agent such as an interleukin 6 targeting agent, and a pharmaceutical composition comprising a chemotherapeutic agent. A preferred chemotherapeutic agent is cisplatin, 5-fluorouracil, or a combination thereof.

### FIGURE LEGENDS

FIG. 1: Boxes indicate median with first and third quartiles of log 2 transformed gene expression values from two U133 Plus 2.0 microarray datasets of pancreatic cancer patients comparing normal (N) and tumor (T) tissue. Badea et al. set (GSE15471) [Badea et al., 2008. Hepato-Gastroenterol 55: 2016-27], n=36 paired biopsies; Pei et al. set (GSE16515) [Pei et al., 2009. Cancer Cell 16: 259-66]; n=16 (normal), n=36 (tumor). ***p<0.001, statistical testing was by two-tailed Student's t test.

FIG. 2: Log 2 transformed ADAM12 expression values from the Pilarksy et al. (E-MEXP-1121) [Pilarsky et al., 2008. J Cell Mol Med 12: 2823-35] gene expression set obtained from micro-dissected pancreatic cancer tissue. *p<0.05, testing by two-tailed Student's t test.

FIG. 3: Transcript levels for indicated mouse Adam 10, 12 and 17 and human ADAM 10, 12 and 17 paralogs relative to Gapdh/GAPDH were measured in xenografts by qPCR using mouse- or human-specific primers. Boxplots show data from 10 individual patient grafts. For each replicate sample measured by qPCR, a technical triplicate was used. Difference between groups was tested by ANOVA for both panels. p<0.0001.

FIG. 4: ADAM12 is elevated in the serum of PDAC patients and predicts poor outcome in patients undergoing resection. ADAM12 levels were measured by ELISA in serum of healthy individuals (n=38), and patients diagnosed with PDAC (n=157). Boxplots show median with interquartile range. ***p<0.0001; tested by Mann-Whitney U-test against healthy controls.

FIG. 5: Stromal CAF-secreted IL-6 drives therapy resistance. (A-C) Cell viability assays were performed on primary 007B cells incubated for 168 h in the following culture conditions; unconditioned medium without chemotherapeutics (untreated), unconditioned medium with chemotherapeutics (control), conditioned medium with chemotherapeutics, medium supplemented with the indicated cytokines and chemotherapeutics (red and blue bars), or 081RF supernatant with or without neutralizing antibodies for the indicated cytokines and chemotherapeutics. Graphs show means±SEM of data normalized to t=0, n=3. P-values were by one-way ANOVA and compared to the control or 081RF (“−”) sup only condition. (D-F) As for panels A-C, using 031M cells. (G) Human IL-6 was measured by ELISA in 3 day-incubated supernatant of the indicated cultures (5 days for 243RF culture) and media not incubated on cells. (H) Mouse IL-6 was measured by ELISA as for panel G in supernatants from indicated (co)cultures. (1) 293T, 007B, and 031M cells were stimulated for 20 minutes with medium containing 3-day incubated 081RF supernatant, diluted 1 in 4. Recombinant IL-6 was used as a positive control and IL-6 neutralizing antibody as negative control for IL-6-induced STAT3 phosphorylation. Following exposure, cells were lysed and processed for Western blot analysis for the indicated antigens.

### DETAILED DESCRIPTION OF THE INVENTION

### Definitions

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” when referring to recited components, elements or method steps also include embodiments which “consist of” said recited components, elements or method steps.

Unless otherwise defined, all terms used in disclosing the concepts described herein, including technical and scientific terms, have the meaning as commonly understood by one of ordinary skill in the art. By means of further guidance, definitions for the terms used in the description are included to better appreciate the teaching of the present disclosure. The terms or definitions used herein are provided solely to aid in the understanding of the teachings provided herein.

The term “tumor”, as used herein, refers to a mass of tissue that is formed by an accumulation of abnormal cells. These abnormal cells divide more than normal cells and/or do not die when cell death is required.

The term “cancer”, as used herein, refers to a group of diseases involving uncontrolled, abnormal cell growth with the potential to invade or spread to other parts of the body.

The term “stroma-rich tumor”, as used herein, refers to a tumor that is characterized by a low tumor-stroma ratio, by the presence of activated stroma, or by a combination thereof. Said low tumor-stroma ratio may be associated with poor clinical outcome (Wu et al., 2016. Oncotarget 7: 68954-68965. Activated stroma may promote tumorigenesis, for example by increasing resistance of cancer cells to anti-cancer therapies (Valkenburg et al., 2018. Nat Rev Clin Oncol 15: 366-381).

The term “stroma”, as used herein, refers to a non-malignant compartment surrounding tumor cells. Stroma may comprise extracellular matrix proteins, cancer-associated fibroblasts and immune cells. Stroma typically forms a vast majority of the tumor's mass and may contribute to resistance to chemotherapy by acting as a barrier to the delivery of chemotherapeutics.

The term “disintegrin and metalloproteinase domain-containing protein 12 (ADAM12)”, as used herein, refers to a member of the “A Disintegrin And Metalloproteases” (ADAM) protein family. ADAM12 comprises extracellular metalloprotease and intracellular signaling properties. ADAM12 is also known as meltrin-alpha, ADAM Metallopeptidase Domain 12, Disintegrin And Metalloproteinase Domain-Containing Protein 12, MLTN, Metalloprotease-Disintegrin 12 Transmembrane, MCMPMltna. ADAM12 is described as UniProt entry 043184. ADAM12 is involved in cell adhesion by binding to integrins and syndecans, as well as in proteolytic cleavage of substrates from producing cells, a process known as ectodomain shedding. Cancer-related substrates of ADAM12 include epidermal growth factor (EGF) and Sonic Hedgehog (SHH).

The term “detecting ADAM12”, as used herein, refers to the detection of ADAM12, or of a part of ADAM12.

The term “bodily fluid”, as used herein, refers to any liquid portion of the body such as milk, blood, synovial fluid, urine, cerebrospinal fluid, bronchiolar lavage fluid, extracellular fluid including lymphatic fluid and transcellular fluid, tear fluid, and/or vitreous humor.

The term “blood”, as used herein, includes reference to blood serum and blood plasma. The terms “blood serum” and “blood plasma” both refer to blood components without cells, whereby blood serum also excludes clotting factors such as fibrinogen. As is known to a person skilled in the art blood may, for example, be centrifuged to remove cellular components. The thus obtained plasma may be coagulated followed by, for example, centrifugation to remove clotting factors, resulting in serum.

The term “stroma-targeting agent”, as used herein, refers to an agent that can influence or modulate stroma that is associated with a tumor, preferably by promoting breakdown of said stroma and/or said tumor. Influencing or modulating stroma associated with a tumor may, for example, be accomplished by inhibiting tumor-promoting signaling between stromal cells, such as cancer-associated fibroblasts, and tumor cells. Said stroma-targeting agent includes a modulator of the renin-angiotensin system such as an angiotensin converting enzyme-inhibitor and an angiotensin II receptor blocker, and an interleukin 6 (IL6) targeting agent.

The term “angiotensin converting enzyme (ACE)-inhibitor”, as is used herein, refers to a group of compounds that block the conversion of angiotensin I to angiotensin II by angiotensin converting enzyme.

The term “angiotensin II receptor blocker (ARB)”, also termed angiotensin II receptor antagonists, as is used herein, to a group of compounds that modulate the renin-angiotensin system by preventing binding of angiotensin II to its receptor and/or subsequent activation of the receptor. There are two angiotensin receptors, termed AT1 and AT2, which both are G protein-coupled receptors that have a sequence identity of about 30% at the amino acid level. The term “interleukin 6 (IL6) targeting agent”, as is used herein, refers to an agent that may influence or modulate stroma interleukin 6 signaling. Influencing or modulating IL6 may, for example, be accomplished by binding to IL6 itself, thereby preventing or blocking an interaction between IL6 and its receptor; by binding to an IL6 receptor, comprising a ligand-binding IL-6Ra chain (CD126), and a signal-transducing component glycoprotein (gp) 130, thereby preventing or blocking an interaction between IL6 and its receptor; or by inhibiting downstream signaling such as by inhibiting a p38 mitogen-activated protein kinase pathway.

Binding to IL6 may be accomplished by administration of an anti-IL6 antibody or by administration of a soluble receptor that binds and sequesters IL6.

Known antibodies that bind to the IL6 receptor include tocilizumab (Hoffmann-La Roche and Chugai), sarilumab (Sanofi and Regeneron Pharmaceuticals), and vobarilizumab (Ablynx NV).

Known antibodies that bind IL6 include olokizumab (UCB Pharma), elsilimomab (Diaclone), clazakizumab (Bristol Myers Squib and Alder Biopharmaceuticals), sirukumab (Janssen Biotech, Inc.), and siltuximab (Janssen-Cilag International NV).

The term “human control”, as used herein, refers to a human person who does not have a cancer that is characterized by at least one stroma-rich tumor.

The term “lymph node ratio”, as used herein, refers to the ratio of the number of metastatic lymph nodes to the total number of examined lymph nodes. A lymph node ratio of more than 0.2 such as more than 0.33, indicates a higher level of metastasis and a worse prognosis, when compared to a lymph node ratio of 0.33 or less, such as a lymph node ratio of 0.2 or less.

The term “esophageal cancer”, as used herein, refers to a cancer that originates from the esophagus. Two main subtypes of esophageal cancer can be distinguished: squamous-cell carcinoma and esophageal adenocarcinoma.

The term “squamous-cell carcinoma”, as used herein, refers to an esophageal cancer that originates from the squamous cells lining the surface of the esophagus. Squamous-cell carcinoma often occurs in the upper and middle portions of the esophagus.

The term “esophageal adenocarcinoma”, as used herein, refers to esophageal cancer that originates from the epithelial tissue with glandular origin, glandular characteristics, or both, in the esophagus. Esophageal adenocarcinoma often occurs in the lower portion of the esophagus.

The terms “treatment”, “treating” and the like, as used herein, generally mean obtaining a desired pharmacologic, a physiologic effect, or both. The effect may be therapeutic in terms of a partial or complete cure for a disease, reduction of adverse effects attributable to the disease, or any combination thereof.

The term “chemotherapy”, as used herein, refers to a cancer treatment method wherein one or more anti-cancer drugs are administered to a patient, this anti-cancer drug affecting cell growth and cell division. Chemotherapy in general affects replication of both cancerous cells and normal cells, often resulting in side effects upon treating a cancer patient with chemotherapy.

The term “surgery”, as used herein, refers to a physical procedure in which cancer-associated cells are removed from the body. Said procedure comprises making incisions in tissue, often skin or tissue aligning internal cavities in a human patient. In esophageal cancer surgery, small tumors can be excised in combination with a margin of healthy tissue surrounding the tumor. Excision of small esophageal tumors can be carried out by passing an endoscope through the throat and into the esophagus. Larger esophageal tumors may require esophagectomy, which comprises the excision of a large part of the esophagus, the upper side of the stomach and nearby lymph nodes, and the reconnection of the remainder of the esophagus to the stomach.

The terms “radiation therapy”, “radiation oncology” and the like, as used herein, refer to a cancer treatment method using ionizing radiation to kill especially cancerous cells. The ionizing radiation is typically X-ray and can be applied either from the outside of the body, or by brachytherapy, wherein radioactive sources are placed at or near a tumor inside a patient. The mode of action of radiation therapy is by causing DNA damage due to either ionization of DNA directly, or by ionization of water causing the formation of free radicals and indirectly damaging DNA.

The term “targeted therapy”, as used herein, refers to a cancer treatment method comprising the administration of molecules that specifically block the growth of cancer cells by interfering with specific targeted molecules which are necessary for carcinogenesis and tumor growth. Targeted therapy differs from chemotherapy in that it specifically targets specific molecules on cancer-related tumor cells, instead of all rapidly dividing cells. The molecules that are used in targeted therapy include monoclonal antibodies and small molecules such as tyrosine-kinase inhibitors. Small molecules are defined as organic compounds with a molecular weight of less than 900 daltons. Targeted therapy with monoclonal antibodies is also an example of “immunotherapy”.

The term “immunotherapy”, as used herein, refers to a cancer treatment method comprising the stimulation of the immune system of the patient to inhibit or kill cancer-associated tumor cells. Immunotherapy can be categorized as active, passive or a combination of active and passive. Active immunotherapy directs the immune system to attack tumor cells directly by targeting antigens displayed on tumor cells. In passive immunotherapy, modified antibodies target antigens displayed on tumor cells, activating any pathway that will result in cell death of the tumor cells.

The term “hormone therapy”, as used herein, refers to a cancer treatment method comprising blocking and/or lowering of a concentration of one or more specific hormones. This can be performed by either blocking the human body's ability to produce said specific hormone or said specific hormones, or by interfering with how specific hormones behave in the human body. Some cancers, such as breast, prostate, ovarian and endometrial cancer require hormone stimulation such as steroid stimulation, to grow and/or develop. Hormone therapy specifically prevents the growing and division of hormone dependent/sensitive cancer cells.

The term “orally administered”, or “per os (p.o.)”, as used herein, refer to the route of administration wherein a compound is taken through the mouth.

The terms “intravenously administered”, “intravenous administration” and the like, as used herein, refer to the route of administration wherein fluids are directly administered into a vein.

The terms “subcutaneously administered”, “subcutaneous administration” and the like, as used herein, refer to the route of administration wherein fluids such as drugs are injected into the subcutis, allowing for a slow, sustained rate of absorption by the blood of the patient.

The terms “intramuscularly administered”, “intramuscular administration” and the like, as used herein, refer to the route of administration wherein fluids such as drugs are injected directly into a muscle.

The term “interleukin 6”, or IL-6, IL6, BSF2, HGF, HSF, IFNB2, BSF-2, CDF, IFN-beta-2, as used herein, refers to an interleukin that acts as both a pro-inflammatory cytokine and an anti-inflammatory myokine. In humans, it is encoded by the IL6 gene (also known as Ensembl: ENSG00000136244 and HGNC: 6018. The encoded protein is known as UniProtKB: p05231.

The term “carbohydrate antigen 19-9 (CA19-9)”, is also known as sialyl-Lewis^{A}, sialyl Lewis(a) tetrasaccharide, 5-acetylneuraminyl-2-3-galactosyl-1-3-(fucopyranosyl-1-4)-N-acetylglucosamine, Neu5Acα2-3Galβ1-3[Fucα1-4]GlcNAcβ, cancer antigen 19-9. It refers to a specific mucin glycoprotein connected with the Lewis a blood group. The exact biological role of CA19-9 is not completely clear, but is suspected to disrupt cell adhesion and promote tumor invasion and metastasis through binding to the cell surface receptors E-selection and P-selectin, which are located on endothelial cells.

The term “quantitative polymerase chain reaction (qPCR)”, also known as real-time polymerase chain reaction (real-time PCR), refers to a technique wherein DNA sequences are exponentially amplified in consecutive cycles while detecting the DNA concentration during the amplification. The detection of DNA can be carried out in several ways, either non-specific, wherein any amplified DNA is detected, or specific, wherein amplification of a specific DNA sequence is detected. An example of non-specific DNA detection are double strength DNA (dsDNA) dyes, such as SYBR Green, which will bind all dsDNA, resulting in an increase in fluorescence of the dsDNA dye upon amplification. Fluorescence can be measured every amplification cycle to monitor total dsDNA concentration. An example of specific DNA detection is detection with a fluorescent reporter probe, comprising of a fluorescent emitter, a probe complementary with a target DNA and, optionally, a quencher of the fluorescent emitter. Upon hybridization with target DNA, said fluorescent emitter become displaced from the quencher and will start to fluorescently emit upon excitation, which can subsequently be detected.

The term “antibody” as used herein, refers to an antigen binding protein comprising at least a heavy chain variable region (Vh) that binds to a target epitope. The term antibody includes monoclonal antibodies comprising immunoglobulin heavy and light chain molecules, single heavy chain variable domain antibodies, and variants and derivatives thereof, including chimeric variants of monoclonal and single heavy chain variable domain antibodies.

### Methods of Treatment

The invention relates to the use of a stroma-targeting agent to treat a human patient having cancer characterized by at least one stroma-rich tumor, said human patient having increased disintegrin and metalloproteinase domain-containing protein 12 (ADAM12) levels in a bodily fluid, when compared to ADAM12 levels in a bodily fluid of a human control. The invention is based, at least in part, on the discovery that ADAM12 is a marker associated with activated stroma surrounding a tumor. Enhanced expression of ADAM12 is associated with poor outcome. Stroma-rich tumors can therefore be identified by measuring ADAM12 levels in a bodily fluid, preferably in blood. Therefore, ADAM12 is a marker for poor prognosis of patients with stroma-rich tumors. Prognosis of stroma-rich tumor patients may further be related to a size of the stroma, whereby a greater size may result in increased ADAM12 levels, to a lymph node ratio, to a level of carbohydrate antigen 19-9, or to a combination thereof.

A level of ADAM12 preferably is expressed in ng/ml. A calibration curve, including known amounts of ADAM12, preferably is included in a method of measuring ADAM12 levels in a bodily fluid, preferably in blood.

A method for determining absence or presence of ADAM12 in bodily fluid, or of a relative level of ADAM12 in a bodily fluid, comprises providing a bodily fluid from a cancer patient, and determining whether said bodily fluid comprises ADAM12 proteins, or parts thereof. These ADAM12 proteins, or parts thereof, may originate from shedding of the cell-bound protein. Detection of ADAM12 proteins, or parts thereof, in a bodily fluid may be determined by any method known in the art, including spectrometry methods such as high-performance liquid chromatography (HPLC), liquid chromatography coupled to mass spectrometry (LC/MS), and antibody dependent methods such as immunoprecipitation, an immunoassay such as enzyme-linked immunosorbent assay (ELISA), immunoelectrophoresis such as Western blotting, and immunostaining.

If required, several methods may be employed to concentrate ADAM12 protein, for example by affinity chromatography or immunoprecipitation using, for example, affinity partners, such as antibodies or functional parts thereof. It is preferred that the affinity partners such as antibodies or functional parts thereof are coupled to a carrier material such as beads, preferably magnetic beads, or to monolithic material, preferably monolithic material that is embedded in columns, preferably in micro-columns.

The affinity partners may be coupled directly to the beads or monolithic material, or indirectly, for example by coupling of a second antibody that specifically recognizes the ADAM12-specific antibody. Said antibodies preferably are indirectly coupled to the beads or monolithic material by coupling of protein A, protein G, or a mixture of protein A and G to the beads or to the monolithic material. Said antibodies, preferably polyclonal antibodies, are preferably coupled to protein A-coupled beads or protein A-coupled monolithic material.

A sample comprising proteins from a bodily fluid, which may comprise ADAM12, may be incubated with the beads or monolithic material under circumstances that allow binding of ADAM12 to the affinity partners on the beads or monolithic material. It is preferred that the proteinaceous sample is repeatedly incubated with the beads or monolithic material under circumstances that allow binding of ADAM12 to the affinity partners. Following the incubation steps, the beads or monolithic material are washed, for example with phosphate-buffered saline or with a 20 mM phosphate buffer at pH 7. Said washing step may also be repeated, preferably 2-20 times, more preferably about 10 times.

Following concentration of ADAM12 by affinity chromatography, the concentrated protein may be released from the affinity partners. Release of ADAM12 may be accomplished by any method known in the art, including the application of a high pH buffer, a low pH buffer and/or a high salt buffer. A preferred elution buffer comprises 200 mM NaOH. Said elution step preferably is repeated, preferably 1-100 times, more preferably 5-20 times, most preferably about 10 times. After collection of the eluate, a buffered solution such as a 50 mM Tris pH 7.9 may be added before further use, rendering the pH<10. Eluates can be stored at −20° C. until further use.

An example of an immunoassay is an enzyme-linked immunosorbent assay (ELISA). ELISA is a commonly used analytical biochemistry assay, wherein a solid-phase enzyme immunoassay is used to detect the presence of a ligand, herein ADAM12 or parts thereof, in a liquid sample, herein in a bodily fluid, preferably blood, using affinity partners such as antibodies which are directed against ADAM12 or parts thereof. An affinity partner such as an antibody that is directed to the detected protein may subsequently be detected by linking the antibody to an enzyme. A substrate for said enzyme may be added, and the resulting reaction produces a detectable signal, commonly a color change.

Suitable ADAM12 ELISA kits are commercially available, for example from Merck (Darmstadt, Germany), R&D Systems (Minneapolis, Minn.) and Abcam (Cambridge, Mass.).

A preferred method for determining presence of ADAM12 in a bodily fluid comprises taking a bodily fluid, preferably blood, from a patient. As is known to a person skilled in the art, plasma may be obtained by removal of cellular components of the blood by centrifugation, precipitation, or a combination of these techniques. Subsequently, a quantitative assay, for example an enzyme-linked immunosorbent assay (ELISA), may be used to detect and quantify ADAM12 in the bodily fluid. ELISA includes 4 different types of methods: direct ELISA, indirect ELISA, sandwich ELISA and competitive ELISA. In direct ELISA, the antigen, here ADAM12, may be immobilized to the surface.

An anti-ADAM12 molecule, conjugated to a molecule that is able to be detected, such as a fluorophore, may be added to bind to ADAM12. Anti-ADAM12 molecules may be antibodies or parts thereof, a single-domain antibody (nanobody), a monoclonal or polyclonal antibody, or any other molecule that is able to bind specifically to ADAM12. In indirect ELISA, ADAM12 may be immobilized to the surface and an anti-ADAM12 molecule may be added. Subsequently, a second molecule may be added, which is able to bind to the anti-ADAM12 molecule, and is conjugated to a molecule that is able to be detected, such as a fluorophore. In sandwich ELISA, an anti-ADAM12 molecule may be immobilized to the surface. Subsequently, a bodily fluid that may comprise ADAM12 is added to the immobilized anti-ADAM12 molecule. A second anti-ADAM12 molecule, which may be conjugated to a molecule that is able to be detected, such as a fluorophore, may be added to bind to ADAM12. Lastly, in competitive ELISA, the concentration of an antigen such as ADAM12 may be detected by signal interference. The sample antigen, ADAM12, may be competing with a reference antigen for a specific binding molecule. A preferred ELISA method of the invention is sandwich ELISA.

Shortly, anti-ADAM12 molecules may be coated onto a surface on which bodily fluid is added. Anti-ADAM12 molecules may be antibodies or parts thereof, a single-domain antibody (nanobody), or any other molecule that is able to bind to ADAM12. A bodily fluid that may or may not contain ADAM12 is subsequently added to the immobilized anti-ADAM12-molecules. An anti-ADAM12 detection molecule may subsequently be added, conjugated to a molecule that may be detected, for example by fluorescence measurements. The anti-ADAM12 detection molecule may be an antibody or a part thereof, a single-domain antibody (nanobody), or any other molecule that is able to bind to ADAM12. Said anti-ADAM12 detection molecule preferably does not compete with the anti-ADAM12 molecule in binding to ADAM12. An example of fluorescent detection consists of an anti-ADAM12 detection molecule conjugated to biotin, followed by addition of horse-radish peroxidase—labeled streptavidin. Lastly, a fluorescent emitter that may be used in ELISA include 3,3′,5,5′-tetramethylbenzidine (CAS 54827-17-7, also known as TMB), which can absorb light at 370 nm and 650 nm, and at 450 nm in an acidic environment. Other examples of detection molecules including a fluorescent or radioactive tag on the anti-ADAM12 detection molecule, are fluorescent dyes of the Alexa Fluor series, fluorescein isothiocyanate (FITC), tetramethylrhodamine (TRITC), rhodamine, Texas Red, or gamma-radioactive isotopes of iodine, such as 125-I, attaced to tyrosine. The intensity of the fluorescent or radioactive label may be compared to a reference intensity, indicating a relative ADAM12 concentration.

Alternatively, ADAM12 may be detected by Western blotting. In Western blotting, proteins are separated based on protein size, molecular weight, isoelectric point, electric charge, or any combination of these factors. Briefly, the protein, herein ADAM12, may be denatured, followed by gel electrophoresis, for example Sodium Dodecyl Sulphate PolyAcrylamide Gel Electrophoresis (SDS-PAGE), to separate ADAM12 from other proteins. Following transfer of the separated proteins to a membrane, the membrane may be incubated with a primary anti-ADAM12 antibody. After washing off excess primary antibody, a secondary antibody that recognizes the primary anti-ADAM12 antibody may be added. Said secondary antibody can be visualized by various methods, such as radiation, fluorescence, luminescence and staining. Quantification of ADAM12 can further be performed by techniques known in the art.

As an alternative, said sample comprising proteins from a bodily fluid, which may comprise ADAM12, may be digested, for example with trypsin, to generate a tryptic digest. Digestion may be performed by application of conditions known to the skilled person. The pH of the obtained solution may be adjusted to 8-8.5, for example with 1 M HCl. Sulphur bridges are preferably reduced, for example by addition of dl-dithiothreitol (DTT). Iodoacetamide is preferably added for methylation of cysteine residues. The digestion time may be between 0.2 and 5 hours. Digestion may be stopped, for example by the addition of formic acid. The digest may be concentrated, for example on an Agilent Bond Elut Plexa SPE column.

Presence or absence of a fragment from ADAM12 may be determined by determining the relative molecular mass of peptides in the sample by a first stage mass spectrometry, after which the peptides may be further fragmented and fragment ions analyzed by second stage of the tandem mass spectrometry. A preferred method for determining a level of ADAM12 comprises ultra-high performance liquid chromatography (UHPLC) coupled to tandem mass spectrometry (LC-MS/MS) in positive electrospray ionization mode, allowing the unambiguous identification and quantification of ADAM12 in a bodily fluid such as blood or serum. The LC-MS/MS analysis may be performed, for example by using a high end UHPLC chromatographic system coupled to a triple-quadrupole mass-spectrometer.

Other indications of the presence of stroma-rich tumors in a human patient include the presence of at least one tumor size of more than 20 mm in diameter, a weight loss of more than 5%, a lymph node ratio of more than 0.2, such as more than 0.33, more than 0.4, or more than 0.5; or a combination thereof.

A tumor size may be determined with a method comprising the steps of: (a) imaging the tumor with esophagogastroscopy, computed tomography (CT), positron emission tomography (PET), magnetic resonance imaging (MRI) or a combination hereof, and (b) determining a largest dimension of the tumor, wherein a length of said largest dimension is used as a proxy for the tumor size. To image the tumor, a contrast agent may be used to generate a clear interpretable image. Examples of contrast agents include radiocontrast agents such as iodinated and barium sulphate contrast agents for imaging with CT, radionuclides including carbon-11, nitrogen-13 and other isotopes with short half-lives for PET imaging, and gadolinium contrast agents and superparamagnetic iron oxide for MRI. Alternatively, methods such as esophagogastroscopy can be used to determine a size of the tumor. These methods do not employ contrast agents. All these techniques allow to form a three-dimensional image of the tumor, thus allowing for determining a largest dimension of the tumor.

A weight loss of more than 5% in a period of time before diagnosis and treatment can easily be determined by a person skilled in the art. A significant weight loss before diagnosis, such as more than 5% in a period of 6 months before diagnosis, more than 5% in a period of 3 months before diagnosis, more than 5% in a period of 2 months before diagnosis, more than 5% in a period of 1 month before diagnosis, might be indicative for the presence of a stroma-rich tumor.

The lymph node ratio may be determined with a method comprising the steps of: (a) evaluating a number of lymph nodes, for example five or more lymph nodes, for presence or absence of cancer cells; and (b) calculating the lymph node ratio by dividing an amount of lymph nodes with cancer cells by a total amount of evaluated lymph nodes. Lymph nodes are ovoid-shaped organs of the lymphatic system and the adaptive immune system. Lymph nodes are a part of the lymphatic system and are widely spread over the whole human body. When tumor cells have metastasized into a lymph node, they may easily spread to different lymph nodes and thereby metastasize over the whole body. For this reason, the ratio of infected lymph to healthy lymph nodes is an important factor to determine a stage of metastasis of a cancer. The human body comprises 500 to 600 lymph nodes, most of these are located in clusters such as in the abdominal areas, the base of limbs and in the neck area. Depending on a location of a tumor, a person skilled in the art is able to determine nearby located lymph nodes, followed by analysis of said lymph nodes for the presence of cancer cells.

Further indices for the presence of stroma-rich tumors in a human patient include bleeding, dysphagia, vomiting, diarrhea, pain, and/or a carbohydrate antigen 19-9 blood concentration of more than 400 kU/L. As is known to a person skilled in the art, bleeding, dysphagia, vomiting, diarrhea, pain typically are indices for the presence of a stroma-rich esophageal tumor, while a carbohydrate antigen 19-9 blood concentration of more than 400 kU/L typically is an index for the presence of a stroma-rich pancreatic tumor.

A concentration of CA19-9 in a bodily fluid can be determined by any method known in the art, including Western blotting, MS, LC-MS, LC-MS/MS, and ELISA. Said concentration may be determined, for example, by an enzyme-linked immunosorbent assay (ELISA) of a bodily fluid of the patient. ELISA is a commonly used analytical biochemistry assay, wherein a solid-phase enzyme immunoassay is used to detect the presence of a ligand, herein CA19-9, in a liquid sample, herein in a bodily fluid, preferably blood, using antibodies which are directed against CA19-9. The antibody directed to the detected protein is subsequently detected, commonly by linking the antibody to an enzyme. A substrate for said enzyme is added, and the resulting reaction produces a detectable signal, commonly a color change. CA19-9 ELISA kits are commercially available, for example from Abcam (Cambridge, Mass.), Thermo Fisher Scientific (Waltham, Mass.), and BioVision (Milpitas, Calif.).

The unit kilounits per liter (kU/L), is an international unit (IU) and standardly used measure for concentrations of antigens.

Said cancer that is characterized by at least one stroma-rich tumor may be a carcinoma, such as a breast carcinoma, colorectal carcinoma, non-small cell lung cancer, breast carcinoma, esophageal squamous cell carcinoma, ovarian cancer, hepatocellular carcinoma, nasopharyngeal cancer, Said cancer especially is a adenocarcinoma, such as a invasive ductal breast carcinoma, pancreatic ductal adenocarcinomas, prostate cancer, cervical cancer, stomach cancer, pancreatic ductal adenocarcinoma and esophageal adenocarcinoma.

The invention further provides a modulator of the renin-angiotensin system as a stroma-targeting agent. Said modulator of the renin-angiotensin system preferably is an angiotensin converting enzyme (ACE)-inhibitor and/or an angiotensin II receptor blocker (ARB).

Said ACE-inhibitor preferably is captopril ((2R)-1-[(2S)-2-methyl-3-sulfanylpropanoyl]pyrrolidine-2-carboxylic acid), which may be administered at a dosage of 5-150 mg daily, preferably at 25-50 mg daily; zofenopril ((2S,4S)-1-[(2S)-3-benzoylsulfanyl-2-methylpropanoyl]-4-phenylsulfanylpyrrolidine-2-carboxylic acid), which may be administered at a dosage of 1-180 mg daily, preferably at 10-60 mg daily; enalapril ((2S)-1-[(2S)-2-[[(2S)-1-ethoxy-1-oxo-4-phenylbutan-2-yl]amino]propanoyl]pyrrolidine-2-carboxylic acid), which may be administered at a dosage of 0.1-50 mg daily, preferably at 2.5-10 mg daily; ramipril ((2S,3aS,6aS)-1-[(2S)-2-[[(2S)-1-ethoxy-1-oxo-4-phenylbutan-2-yl]amino]propanoyl]-3,3a, 4,5,6,6a-hexahydro-2H-cyclopenta[b]pyrrole-2-carboxylic acid), which may be administered at a dosage of 0.1-50 mg daily, preferably at 2.5-20 mg daily; quinapril ((3S)-2-[(2S)-2-[[(2S)-1-ethoxy-1-oxo-4-phenylbutan-2-yl]amino]propanoyl]-3,4-dihydro-1H-isoquinoline-3-carboxylic acid), which may be administered at a dosage of 1-160 mg daily, preferably at 20-80 mg daily; perindopril ((2S,3aS,7aS)-1-[(2S)-2-[[(2S)-1-ethoxy-1-oxopentan-2-yl]amino]propanoyl]-2,3,3a,4,5,6,7,7a-octahydroindole-2-carboxylic acid), which may be administered at a dosage of 0.2-50 mg daily, preferably at 2-8 mg daily; lisinopril ((2S)-1-[(2S)-6-amino-2-[[(1S)-1-carboxy-3-phenylpropyl]amino]hexanoyl]pyrrolidine-2-carboxylic acid), which may be administered at a dosage of 1-80 mg daily, preferably at 20-40 mg daily; benazepril (2-[(3S)-3-[[(2S)-1-ethoxy-1-oxo-4-phenylbutan-2-yl]amino]-2-oxo-4,5-dihydro-3H-1-benzazepin-1-yl]acetic acid), which may be administered at a dosage of 1-160 mg daily, preferably at 20-40 mg daily; imidapril ((4S)-3-[(2S)-2-[[(2S)-1-ethoxy-1-oxo-4-phenylbutan-2-yl]amino]propanoyl]-1-methyl-2-oxoimidazolidine-4-carboxylic acid; hydrochloride), which may be administered at a dosage of 0.1-100 mg daily, preferably at 2.5-10 mg daily; trandolapril ((2S,3aR,7aS)-1-[(2S)-2-[[(2S)-1-ethoxy-1-oxo-4-phenylbutan-2-yl]amino]propanoyl]-2, 3,3a, 4,5,6,7,7a-octahydroindole-2-carboxylic acid), which may be administered at a dosage of 0.1-10 mg daily, preferably at 0.5-5 mg daily; cilazapril which may be administered at a dosage of 0.1-50 mg daily, preferably at 0.5-5 mg daily; fosinopril ((2S,4S)-4-cyclohexyl-1-[2-[[(1S)-2-methyl-1-propanoyloxypropoxy]-(4-phenylbutyl)phosphoryl]acetyl]pyrrolidine-2-carboxylic acid), which may be administered at a dosage of 1-100 mg daily, preferably at 5-40 mg daily; arfalasin ((2S)-2-[[(2S)-1-[(2S)-2-[[(2S)-2-[[(2S)-2-[[(2S)-2-[[(2S)-2-[(4-amino-4-oxobutanoyl)amino]-5-(diaminomethylideneamino)pentanoyl]amino]-3-methylbutanoyl]amino]-3-(4-hydroxyphenyl)propanoyl]amino]-3-methylbutanoyl]amino]-3-(1H-imidazol-5-yl)propanoyl]pyrrolidine-2-carbonyl]amino]-2-phenylacetic acid), which may be administered at a dosage of 10-2000 mg daily, preferably at 50-500 mg daily; or a combination thereof.

Said angiotensin II receptor blocker (ARB) preferably is valsartan ((2S)-3-methyl-2-[pentanoyl-[[4-[2-(2H-tetrazol-5-yl)phenyl]phenyl]methyl]amino]butanoic acid), which may be administered at a dosage of 10-1000 mg daily, preferably at 40-160 mg daily; telmisartan (2-[4-[[4-methyl-2-propyl-6-[1-(trideuteriomethyl)benzimidazol-2-yl]benzimidazol-1-yl]methyl]phenyl]benzoic acid), which may be administered at a dosage of 5-200 mg daily, preferably at 40-80 mg daily; losartan ([2-butyl-5-chloro-3-[[4-[2-(2H-tetrazol-5-yl)phenyl]phenyl]methyl]imidazol-4-yl]methanol), which may be administered at a dosage of 1-500 mg daily, preferably at 25-100 mg daily; irbesartan (2-butyl-3-[[4-[2-(2H-tetrazol-5-yl)phenyl]phenyl]methyl]-1,3-diazaspiro[4.4]non-1-en-4-one; hydrochloride), which may be administered at a dosage of 10-500 mg daily, preferably at 150-300 mg daily; azilsartan (2-ethoxy-3-[[4-[2-(5-oxo-4H-1,2,4-oxadiazol-3-yl)phenyl]phenyl]methyl]benzimidazole-4-carboxylic acid), which may be administered at a dosage of 5-500 mg daily, preferably at 20-80 mg daily; olmesartan (5-(2-hydroxypropan-2-yl)-2-propyl-3-[[4-[2-(2H-tetrazol-5-yl)phenyl]phenyl]methyl]imidazole-4-carboxylic acid), which may be administered at a dosage of 1-100 mg daily, preferably at 10-40 mg daily; or a combination thereof.

A preferred modulator of the renin-angiotensin system is or comprises losartan.

The invention further provides an interleukin 6 (IL-6)-targeting agent as a stroma-targeting agent. Said IL-6 targeting agent comprises tocilizumab (also known as atlizumab, CAS no. 375823-41-9, MRA, R-1569), which may be administered at a dosage of 100-1000 mg once every week; siltuximab (also known as CAS no. 541502-14-1), which may be administered at a dosage of 11 mg/kg every 1-3 weeks; olokizumab (CAS no. 1007223-17-7), which may be provided at a dosage of 64 mg, once per 4 weeks; elsilimomab (also known as B-E8, CAS no. 468715-71-1), which may be provided at a dosage of 5-50 mg once a day; clazakizumab (also known as ALD518, BMS-945429 or CAS no. 1236278-28-6), which may be provided at a dosage of 25-200 mg once per month; sirukumab (also known as CNTO-136 or CAS no. 1194585-53-9), which may be provided at a dosage of 50-100 mg every 2-4 weeks; sarilumab (also known as Kevzara, CAS no. 1189541-98-7), which may be provided at a dosage of 200 mg every two weeks; vobarilizumab (CAS no. 1628814-88-9), which may be provided at a dosage of 75-225 mg, every 2 to 4 weeks, or a combination thereof.

A stroma-targeting agent is preferably administered orally, intravenously, subcutaneously, intramuscularly or a combination thereof, most preferably orally, intravenously by infusion, or subcutaneously.

Said modulator of the renin-angiotensin system, such as an angiotensin converting enzyme (ACE)-inhibitor and/or an angiotensin II receptor blocker (ARB), preferably is administered orally, for examples as a capsule or tablet. Some modulators may be provided as a prodrug, which is converted to its active metabolite after administration. For example, perindopril is hydrolyzed to its active metabolite perindoprilat in the liver.

When administrating intravenously, a stroma-targeting agent may be directly delivered into a vein. This can be performed via injection, using a syringe under an elevated pressure, or via an infusion, which typically uses gravity to create an increased pressure in order to transfer the agent to the vein. Intravenous infusions are also called “drips”. Intravenous administration delivers the agent directly into the bloodstream via the vein. Said agent is thus rapidly distributed over the whole body, faster than when administered orally. The point of administration in the body can either be in the peripheral lines, such as arms, hands, legs and feet, or in the central lines, such as in the torso. The stroma-targeting agent is preferably administered in a peripheral line over a central line.

When administrating subcutaneously, a stroma-targeting agent is delivered to the subcutis. The agent may be injected into the outer area of the upper arm, the abdomen, the front of the thigh, the upper back, or the upper area of the buttock, just behind the hip bone, depending on the desired absorption rate of the stroma-targeting agent. The agent is absorbed in a steady, slow rate compared to intravenous administration. The agent may be administered at either 90 degrees or 45 degrees to the skin surface. The length of the needle may be adjusted depending on the angle and the amount of subcutaneous tissue present.

When administrating intramuscularly, a stroma-targeting agent may be delivered into a muscle. As the muscles have more blood vessels than subcutaneous tissue, the agent is delivered faster to the blood stream, when compared to subcutaneous administration. The agent can be injected into various possible muscles, for example the deltoid, dorsogluteal, rectus femoris, vastus lateralis and vetrogluteal muscles. Only a small volume is administered at a time, for example between 2 and 5 milliliters and depended on the injection site. The syringe needle is inserted in the skin at an angle between 72 and 90 degrees with a rapid motion in order to cause less discomfort, followed by slowly inserting the agent by pushing on the plunger of the syringe.

The invention further provides a method for treating a human patient having a cancer characterized by at least one stroma-rich tumor, said human patient having increased disintegrin and metalloproteinase domain-containing protein 12 (ADAM12) levels in a bodily fluid, when compared to ADAM12 levels in a bodily fluid of a human control, the method comprising treating the human patient with a stroma-targeting agent, wherein the method further comprises treatment with a chemotherapeutic compound. Possible therapeutic compounds include a monoclonal antibody such as trastuzumab (also known as anti-c-erB-2, anti-ERB-2, CAS no 180288-69-1).

Trastuzumabl binds to the extracellular domain of the human epidermal growth factor receptor 2 protein (HER-2) and mediates antibody-dependent cellular cytotoxicity by inhibiting proliferation of cells which overexpress HER-2 protein. Said antibody may be administered at a dosage of 2-8 mg/kg infused over 90 minutes, every one to three weeks; Further therapeutic compounds include chemotherapeutical agents such as cytotoxic agents, immunomodulating agents and immunotoxic agents. Said one or more chemotherapeutical agents include alkylating agents such as busulfan, melphalan, carboplatin, cisplatin, cyclophosphamide, dacarbazine, carmustine, nimustin, lomustine, ifosfamide, temozolomide, navelbine and altretamine, antibiotics such as leomycin, doxorubicin, adriamycin, idarubicin, epirubicin and plicamycin, antimetabolites such as sulfonamides, folic acid antagonists, gemcitabine, 5-fluorouracil (5-FU), leucovorine, leucovorine with 5-FU, 5-FU with calcium folinate and leucovorin, capecitabine, mercaptopurine, cladribine, pentostatine, methotrexate, raltitrexed, pemetrexed, thioguanine, and camptothecin derivates such as topotecan and irinotecan, hormones and antagonists thereof such as flutamide, goserelin, mitotane and tamoxifen, mustard gas derivatives such as melphalan, carmustine and nitrogen mustard, and alkaloids such as taxanes, docetaxel, paclitaxel, etoposide, vincristine, vinblastine and vinorelbine.

Preferred agents include paclitaxel (also known as (2α,4α,5β,7β,10β,13α)-4,10-Bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate), which may be provided at a dosage of 135-175 mg/m² over 3-24 hours every 1-3 weeks; cisplatin (also known as CDDP, cis-DDP, cis-Diamminedichloroplatinum, cis-platinum, or (SP-4-2)-diamminedichloroplatinum(II)), which may be administered at a dosage of 40-100 mg/m² every 1 to 4 weeks; a fluoropyrimidine such as 5-fluorouracil (also known as fluorouracil, 5-fluracil, 5-FU, 5FU, or 5-fluoro-1H,3H-pyrimidine-2,4-dione), which may be administered at a dosage of 200-1000 mg/m²/day as a continuous infusion over 24 hours, preferably every 3 weeks, and capecitabine (pentyl N-[1-[(2R,3R,4S,5R)-3,4-dihydroxy-5-methyloxolan-2-yl]-5-fluoro-2-oxopyrimidin-4-yl]carbamate), which may be administered at a dosage of 600-1000 mg/m²/day as a continuous infusion over 24 hours, preferably for at least two weeks; dosage of a fluoropyrimidine may depend on dihydropyrimidine dehydrogenase genotype (Henricks et al., 2018. Lancet Oncol 19: 1459-67); oxaliplatin (also known as eloxatin, L-OHP, [(1R,2R)-cyclohexane-1,2-diamine](ethanedioato-O,O′)platinum(II)), which may be administered at a dosage of 20-130 mg/m² every 2 to 3 weeks; irinotecan ([(19S)-10,19-diethyl-19-hydroxy-14,18-dioxo-17-oxa-3,13-diazapentacyclo[11.8.0.02,11.04,9.015,20]henicosa-1(21),2,4(9),5,7,10,15(20)-heptaen-7-yl]4-piperidin-1-ylpiperidine-1-carboxylate), which may be administered at a dosage of 10-1000 mg/m²/day, such as 350 mg/m²/day, as a continuous infusion over 30-90 minutes, preferably every 3 weeks, carboplatin (also known as CBDCA, cis-diammine(cyclobutane-1,1-dicarboxylate-O,O′)platinum(II)), which may be administered at a dosage of 250-450 mg/m² every 2 to 4 weeks; ramucirumab (also known as CAS no 947687-13-0), which may be administered at a dosage of 1-20 mg/kg, preferably 8-10 mg/kg every 2 weeks; folinic acid (also known citrovorum factor, leucovorin, 5-formyltetrahydrofolate, (2S)-2-{[4-[(2-amino-5-formyl-4-oxo-5,6,7,8-tetrahydro-1H-pteridin-6-yl)methylamino]benzoyl]amino} pentanedioic acid), which may be administered at a dosage of 0.1-10 mg/day, preferably about 1 mg per day, a combination of trifluridine (1-[(2R,4S,5R)-4-hydroxy-5-(hydroxymethyl)oxolan-2-yl]-5-(trifluoromethyl)pyrimidine-2,4-dione) with tipiracil (5-chloro-6-[(2-iminopyrrolidin-1-yl)methyl]-1H-pyrimidine-2,4-dione; hydrochloride), which may be administered at a dosage of 10-160 mg/m²/day, preferably about 35 mg/m² twice daily, or a combination thereof. Said treatment additionally may include an immune checkpoint inhibitor such a PD1/PDL1 inhibitor and/or antibodies against CTLA-4. Suitable immune checkpoint inhibitors are PD1/PDL1 inhibitors such as antibodies, including pembrolizumab (Merck), nivolumab (Bristol-Myers Squibb), pidilizumab (Medivation/Pfizer), MEDI0680 (AMP-514; AstraZeneca) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), BMS-936559 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl]methyl]piperidine-2-carboxylic acid), BMS202 (PD-1/PD-L1 Inhibitor 2; WO2015034820; N-[2-[[[2-methoxy-6-[(2-methyl[1,1′-biphenyl]-3-yl)methoxy]-3-pyridinyl]methyl]amino]ethyl]-acetamide), and PD-1/PD-L1 Inhibitor 3 (WO/2014/151634; (3S,6S,12S,15S,18S,21S,24S,27S,30R,39S,42S,47a5)-3-((1H-imidazol-5-yl)methyl)-12,18-bis((1H-indol-3-yl)methyl)-N, 42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4, 7,10,13,).

Treatment of the patient may additionally comprise surgery, chemotherapy, radiation therapy, targeted therapy, immunotherapy, hormone therapy or a combination thereof.

When the patient is further treated with surgery, a part of a tumor, preferably a complete tumor comprising substantially all tumor cells, may be excised from the patient, together with a small margin of healthy cells surrounding the tumor. In the case of metastasis, surgery will not be sufficient and will need to be assisted with other treatment methods.

Said other treatment methods can be performed before or after surgery, being neoadjuvant or adjuvant therapy, respectively. Neoadjuvant therapy is used to reduce the initial tumor size to facilitate subsequent surgery and providing surgery a higher chance of completely excising the tumor. Adjuvant therapy is applied after surgery, and is usually provided when all detectable tumor cells are removed, but when a statistical risk of relapse remains, due to the presence of undetectable tumor cells.

When a patient is further treated with chemotherapy, it is often applied as neoadjuvant therapy, adjuvant therapy or both therapies. Targeted therapy, immunotherapy, and hormone therapy are preferred above chemotherapy whenever possible, as it results in less side effects. The main side effects of chemotherapy are due to the chemotherapy drugs affecting healthy fast growing cells as well as tumor cells. Targeted therapy is possible whenever a marker is known to which a target agent is available, such as monoclonal antibodies in the case of immunotherapy. Hormone therapy is possible when the specific type of cancer requires hormone stimulation to grow and/or develop, such as breast and prostate cancer. Chemotherapy, targeted therapy, immunotherapy, hormone therapy or a combination thereof may be applied before, during or after treatment of a human patient with a stroma-targeting agent. Any of these therapies, or a combination thereof, may be applied as adjuvant, neoadjuvant or both as adjuvant and as neoadjuvant.

When a patient is further treated with radiation therapy, said radiation therapy is often applied at the beginning of a treatment, thus before surgery and before, during or after neoadjuvant therapy. Alternatively, radiation therapy can be applied after surgery to prevent tumor reoccurrence. Radiation therapy may be intended for curative, adjuvant, neoadjuvant or palliative therapy. Radiation therapy may be applied before or after treating the human patient with a stroma-targeting agent.

The invention further provides a pharmaceutical composition comprising a stroma-targeting agent for treating a human patient diagnosed with cancer characterized with at least one stroma-rich tumor, and one or more acceptable excipients.

Said pharmaceutical composition preferably is for use in a method of treating a patient suffering from a stroma-rich tumor, such as an esophageal carcinoma.

Said pharmaceutical composition comprises a stroma-targeting agent for treating a human patient diagnosed with cancer characterized with at least one stroma-rich tumor, and one or more pharmaceutically acceptable excipients. The stroma-targeting agent may be provided to the human patient in such a way that uptake of the stroma-targeting agent is facilitated and possibly enhanced. Additionally, the composition may contain preservatives in order to store the composition with the stroma-targeting agent for a longer time. Ways to increase the uptake of the stroma-targeting agent by modifying the composition include the addition of binders, granulating ingredients and diluents, as it is easier to formulate the correct dose from a larger volume, by reducing the viscosity of the composition, and/or by enhancing the solubility of the stroma-targeting agent.

Pharmaceutically acceptable excipients include diluents, binders or granulating ingredients, a carbohydrate such as starch, a starch derivative such as starch acetate and/or maltodextrin, a polyol such as xylitol, sorbitol and/or mannitol, lactose such as a-lactose monohydrate, anhydrous a-lactose, anhydrous 6-lactose, spray-dried lactose, and/or agglomerated lactose, a sugar such as dextrose, maltose, dextrate and/or inulin, or combinations thereof, glidants (flow aids) and lubricants to ensure efficient tableting, and sweeteners or flavors to enhance taste.

Said pharmaceutical composition preferably comprises a stroma-targeting agent and a compound selected from trastuzumab, paclitaxel, cisplatin, 5-fluorouracil, oxaliplatin, carboplatin, ramucirumab, pembrolizumab, folinic acid, or a combination thereof. Said pharmaceutical composition is for simultaneous, separate or sequential use in the treatment of a stroma-rich tumor in a human individual. Said pharmaceutical composition may comprise a pharmaceutical composition comprising a stroma-targeting agent and a pharmaceutical composition comprising a compound selected from trastuzumab, paclitaxel, cisplatin, 5-fluorouracil, oxaliplatin, carboplatin, ramucirumab, pembrolizumab, folinic acid, or a combination thereof.

A preferred stroma-targeting agent is an interleukin 6 targeting agent, preferably tocilizumab.

### EXAMPLES

### Example 1: ADAM12 is a Circulating Marker for Stromal Activation in Pancreatic Cancer and Predicts Response to Chemotherapy

Materials and Methods

### Collection of Blood Samples

In the AMC cohort, serum samples were obtained perioperatively from 60 patients undergoing resection, or before the start of treatment in case of unresectable patients (n=89). Clinicopathological data were obtained from medical records and included age, gender, tumor diameter, differentiation grade, lymph node ratio (positive lymph nodes/total number of lymph nodes examined), therapies received, and tumor-node-metastasis (TNM) staging. Collection of material was approved by the institute's ethics committee (IRB), and written informed consent was received from all participants (AMC METC 2014_181) [Damhofer et al., 2015. J. Transl. Med. 13:115]. Blood samples of 38 non-age matched healthy individuals without any indication of malignancy were collected as a control group. In the MPACT trial cohort, collection of plasma samples for biomarker development was optional and separate written consent was obtained for sample collection and biomarker analysis. The patients and methods of the MPACT trial have been described previously (trial registration number NCT00844649) [Von Hoff, et al., 2011. J. Clin. Oncol. 29:4548-54]. All patients provided informed consent. In brief, patients were ≥18 years of age, had confirmed measurable metastatic adenocarcinoma of the pancreas, a Karnofsky performance status (KPS) of ≥70, and did not receive prior chemotherapy for metastatic disease. Patients were randomized 1:1 (stratified by performance status, presence of liver metastases, and region) to receive nab-paclitaxel 125 mg/m² plus gemcitabine 1,000 mg/m² on days 1, 8, and 15 every 28 days or gemcitabine alone 1,000 mg/m² on days 1, 8, 15, 22, 29, 36, and 43 in 56 days (cycle 1) and then on days 1, 8, and 15 every 28 days (cycle≥2). Treatment continued until disease progression or unacceptable toxicity.

### Establishment of Patient-Derived Xenografts and Primary Cell Lines

Collection of tumor material was approved by the institute's ethics committee in accordance with the Declaration of Helsinki (AMC METC 2014_181). Written informed consent was received from all participants [Damhofer et al., 2015. J. Transl. Med. 13:115]. All PDAC patients treated in the Academic Medical Center were diagnosed by pathology or cytology. Specimens were processed and inspected according to national and international guidelines. An experienced pathologist performed microscopic assessment. Final diagnosis was set in accordance with the WHO classification, and the pTNM classification of malignant tumors. Freshly excised tumor pieces (approximately 3×3×3 mm) originating from the primary tumor or liver metastasis were washed several times in PBS containing 10 μg/ml gentamycin (Lonza, Basel, Switzerland) and 1% penicillin-streptamycin. Pieces were grafted subcutaneously into the flank of immunocompromised NOD.Cg-Prkdc^{scid} Il2rg^{tm1Wjl}/SzJ (NSG) mice (JAX 005557) with Matrigel (BD, Franklin Lakes, N.J.). Both sexes were used, and ages ranged up to 9 months. Animals were bred and maintained at the local animal facility according to the pertinent legislation, and ethical approval was obtained for all procedures (DTB/LEX102348). After outgrowth to a size of approximately 500 mm³, tumors were harvested and passaged, and/or used to establish in vitro cultures. For this, harvested xenografts were minced with a scalpel blade, placed in IMDM with 8% FBS and collagenase IV (0.5 mg/ml, Sigma-Aldrich, St. Louis, Mo.) and incubated at 37° C. for 60 min with vortexing every 15 min. The dissociated suspension was passed through a 70 μm cell strainer, washed with culture medium and grown in IMDM containing 8% FBS and 50 μM β-mercaptoethanol. During the first 5-10 passages, cultures contained colonies of human epithelial cells and a layer of murine fibroblasts. A culture without epithelial component, as confirmed by flow cytometry using an anti-EPCAM antibody (DAKO, F0860 at 1:100), was used for stimulation experiments. STR profiling was performed to confirm donor-cell line matching (August 2017). For transcript analysis, all eligible and available xenografts were analyzed. Randomization and blinding did not apply. Xenografts were excluded if histopathological assessment did not confirm diagnosis of PDAC.

### RNA Isolation and Quantitative Real-Time PCR

Small pieces of PDX tumor were homogenized using an Ultra-Turrax tissue homogenizer T8 (IKA-Werke, Staufen im Breisgau, Germany) in 1 ml of Trizol (ThermoFisher). Primary cells were lysed in Trizol and RNA isolation was performed according to the manufacturer's protocol. Snap frozen patient tumor samples were embedded in Tissue-Tek OCT (Sakura FineTek, Japan) and 30 slices of 20 μm each were cut on a cryotome. Cut tissue was immersed in 1 ml of RNA Bee (Amsbio, Abingdon, United Kingdom), homogenized, and RNA isolation was performed according to manufacturer's protocol (Qiagen, Hilden, Germany). For tumor percentage scoring, a 10 μm slice was kept before the tissue was cut in 20 μm slices, and processed for H&E staining. Scoring of tumor percentage was performed by an experienced pathologist. cDNA was synthesized using Superscript III (ThermoFisher) and random primers. Real-time quantitative RT-PCR was performed with SYBR green (Roche, Basel, Switzerland) on a Lightcycler LC480 II (Roche). Relative expression of genes was calculated using the comparative threshold cycle (Cp) method and values were normalized to reference gene GAPDH/Gapdh. Primer sequences are:

hGAPDH Fw
5′ gaaggtgaaggtcggagtc 3′;
hGAPDH Rv
5′ tggaagatggtgatgggatt 3′;
hADAM10 Fw
5′ ttcgatgcaaatcaaccaga 3′;
hADAM10 Rv
5′ ttccttcccttgcacagtct 3′;
hADAM12 Fw
5′ tttccaccaccctctcagac 3′;
hADAM12 Rv
5′ gcctctgaaactctcggttg 3′;
hADAM17 Fw
5′ gggaacatgaggcagtctct 3′;
hADAM17 Rv
5′ accgaatgctgctggatatt 3′;
hACTA2 Fw
5′ caaagccggccttacagag 3′;
hACTA2 Rv
5′ agcccagccaagcactg 3′;
hFAP Fw
5′ tcagtgtgagtgctctcattgtat 3′;
hFAP Rv
5′ gctgtgcttgccttattggt 3′;
hSPARC Fw
5′ gaaagaagatccaggccctc 3′;
hSPARC Rv
5′ cttcagactgcccggaga 3′;
hKRT19 Fw
5′ cctggagttctcaatggtgg 3′;
hKRT19 Rv
5′ ctagaggtgaagatccgcga 3′;
mGapdh Fw
5′ ctcatgaccacagtccatgc 3′;
mGapdh Rv
5′ cacattgggggtaggaacac 3′;
mAdam10 Fw
5′ aagatggtgttgccgacagt 3′;
mAdam10 Rv
5′ tggtcctcatgtgagactgc 3′;
mAdam12 Fw
5′ gctttggaggaagcacagac 3′;
mAdam12 Rv
5′ cgcatcaacgtcttcctttt 3′;
mAdam17 Fw
5′ gtacagcgtgaagtggcaga 3′;
mAdam17 Rv
5′ gccccatctgtgttgattct 3′.

### Gene Set Enrichment Analysis (GSEA) and Expression Analysis

Gene set enrichment analysis (GSEA, v2.0.14) software was downloaded from the Broad Institute website (http://www.broadinstitute.org/gsea) and used according to the author's guidelines [Subramanian et al., 2005. Proc Natl Acad Sci USA 102:15545-50]. Median ADAM12 expression was used to dichotomize samples. Gene set for the GO term ‘extracellular matrix’ was downloaded from the Molecular Signature Database (MSigDB; V4.0); the pancreatic stroma signature was published by Binkley et al. [Binkley et al., 2004. Pancreas. 29:254-63]. 2000 phenotype permutations were used to determine significance of the enrichment score. Gene expression data were collected and processed for use in the AMC in-house R2 Genomics Analysis and Visualization Platform: (http://r2.amc.nl). For visualization of gene expression, data were imported in R and plotted using ggplot2, or plotted in Graphpad Prism.

### Treatment of Cultures

Stellate cells (from the Kocher lab [Kadaba et al., 2013. J. Pathol. 230:107-17]) were seeded in 12 well culture plates and upon reaching confluence, pre-starved overnight in 0.5% FBS containing medium and treated for 24 h with 5 ng/ml recombinant human TGF-61 (Peprotech, London, United Kingdom) in the presence or absence of 1 μM ALK4/5/7 inhibitor A83-01 (Tocris Bioscience, Bristol, United Kingdom). ShhN supernatant from 293T cells was added 1:4, EGF was used at 50 ng/ml, bFGF, 10 ng/ml; HGF 10 ng/ml; IL-la, 10 ng/ml; IL-16, 10 ng/ml. Treatment was not randomized between plate wells, and experiments were not blinded. Cultures were STR authenticated (June 2016), and tested for mycoplasma monthly.

### ELISA Analysis of Serum Samples (AMC Cohort)

For the reporting on association of ADAM12 levels with prognosis, the pertinent guidelines were considered [McShane L M et al., 2005. J. Natl. Cancer Inst. 97:1180-4]. Serum was obtained by centrifugation of blood for 10 min at room temperature at 1300 g, and storage at −80° C. until analysis. ADAM12 levels were determined using the human ADAM12 DuoSet ELISA kit (R&D Systems, Minneapolis, Minn.), according to manufacturer's recommendations. Briefly, after coating the 96-well plates (Greiner Nunc MaxiSorp, Kremsmunster, Austria) with capture antibody overnight at room temperature, blocking the plate with 1% BSA the following day, 801 of serum samples were added. After incubation for two hours at room temperature and mild washing steps, samples were incubated with biotinylated detection antibody for additional two hours followed by a 20 min incubation step with horse-radish peroxidase (HRP)-labeled streptavidin. Substrate was tetramethylbenzidine substrate solution (TMB), added for an additional 20 min. Absorbance was measured at 450 nm and 570 nm with a microplate reader (BioTek Synergy BioTek, Winooski, Vt.) after addition of 1M H₂SO₄ stop solution. For wavelength correction, 570 nm readings were subtracted from the 450 nm values before further analysis. The technician performing the analysis was blinded for survival time. Randomization did not apply.

### ELISA Analysis of Plasma Samples (MPACT Cohort)

MPACT trial blood samples were collected in EDTA tubes to chelate calcium and prevent blood clotting, and storage at −80° C. until analysis. To allow the analysis of these plasma samples using the ADAM12 DuoSet ELISA, recalcification was required. 100 μl of EDTA plasma was complemented with 12 mM CaCl₂ (final concentration) and incubated overnight at 4° C. The following day, clots were removed manually and 50 μl sample was used for the assay. The ELISA was performed as for the serum samples. Analysis was blinded to the technician performing the ELISA, who was unaware of the trial arm the samples were from. Sensitivity of the assay was reduced compared to measurement in serum by a factor of 0.44, explaining why in the MPACT cohort, the lowest quartile was composed of samples that had undetectable plasma ADAM12. Measurement of ADAM12 in matched serum and plasma samples revealed high correlation (R²=0.9971, p-value: 2.0×10⁻⁶, n=6).

### Statistics

Student's t-tests were performed using GraphPad Prism 5 software (GraphPad, La Jolla, Calif.). R was used for linear regression analysis of gene expression. All tests were two-sided and p<0.05 was considered statistically significant. Variance was assumed to be similar between compared groups. Adjustments for multiple comparisons were applied for gene correlations across the genome (FDR). For the cell culture experiments, no sample size calculation was performed. For the AMC cohort patient data, SPSS package 24 (IBM Analytics, Armonk, N.Y.) was used for Chi-square testing, Mann-Whitney U test, Spearman's rank correlation, Cox proportional-hazard regression modeling, Kaplan-Meier survival analysis and log-rank test. Patients who died within 30 days after operation were excluded from survival analysis as these likely did not succumb to cancer (n=5). Otherwise, all eligible samples were measured and no power calculation was performed. Cox proportional hazard regression was used for univariate and multivariate analyses to investigate the correlation of OS with ADAM12 and potential prognostic factors.

For the MPACT cohort, baseline ADAM12 values were categorized into undetectable (0) and detectable (>0). The fold change of ADAM12 at cycle 2, day 1 (C2D1) from baseline (cycle 1, day 1; C1D1) was calculated by the results at C2D1 divided by the value at C1D1. These were then assigned to three groups (0: both values below detection limit; <1 if levels decreased, >1 if values increased). Descriptive statistics summary of demographics and baseline characteristics were performed. Correlation of ADAM12 groups with categorical variables was tested using CMH statistics or Cochran-Armitage Trend Test statistics, or one-way ANOVA for continuous variables. All eligible samples were measured and no power calculation was performed. Overall survival was analyzed using Kaplan-Meier method and log-rank test. SAS 9.2 (SAS Institute Inc., Cary, N.C.) was used for all statistical analyses in the MPACT study.

### Results

ADAM12 Associates with Activated Pancreatic Cancer Stroma and Poor-Prognosis Molecular Subclasses

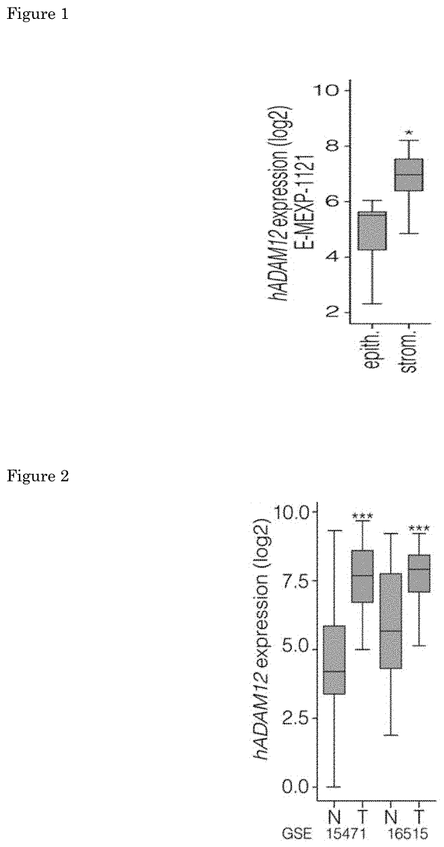

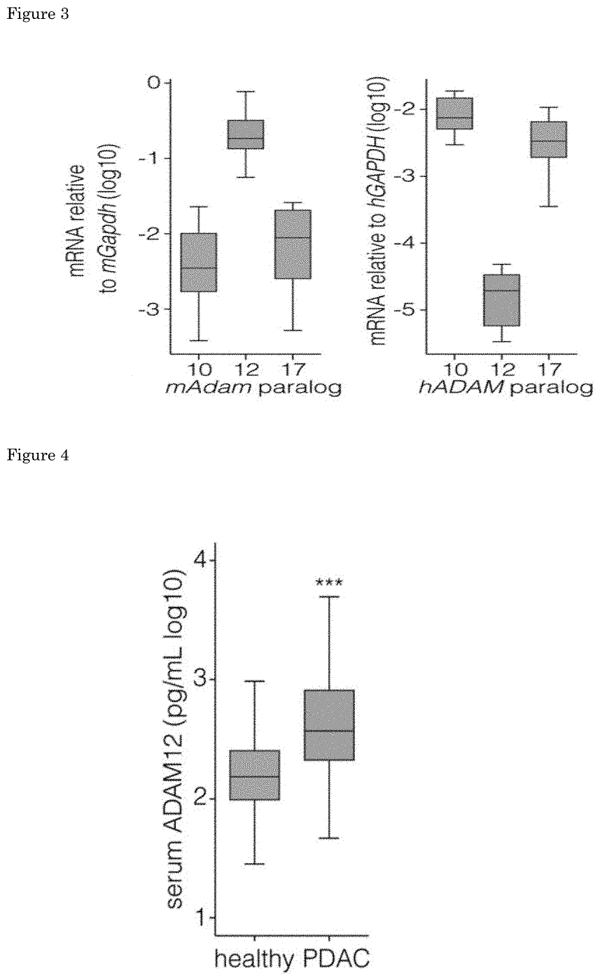

We identified ADAM12 in a previous screen for stromal targets of tumor-derived SHH [Damhofer et al., 2013. Mol. Oncol. 7:1031-42]. To confirm that ADAM12 is expressed in human pancreatic cancers, we queried publically available gene expression datasets that contain normal pancreas and pancreatic cancer tissue. ADAM12 was significantly higher expressed in tumor tissue (FIG. 1), and high expression of ADAM12 was associated with worse survival following resection (data not shown) [Perez-Mancera et al., 2012. Nature 486:266-70; Zhang et al., 2013. Cancer Res. 19:4983-93]. Microdissected tumor tissue expression data confirmed a predominantly stromal expression of ADAM12 (FIG. 2) [Pilarsky et al., 2008. J. Cell. Mol. Med. 12:2823-35]. To further delineate the source of ADAM12 expression, we measured its expression by species-specific qPCR in patient-derived xenografts (PDXs) [Damhofer et al., 2015. J. Transl. Med. 13:115]. Mouse Adam12 expression in stromal host cells was found to be high compared to other well-characterized paralogs (Adam10 and -17; FIG. 3) [Damhofer et al., 2015. J Cell Sci, 128.1: 129-139].

To determine if ADAM12 expression is a hallmark of tumors with highly activated stromal stellate cells and (myo)fibroblasts, its correlation with known markers for such cells was determined by qPCR in bulk tumor tissue (data not shown). A strong correlation of ADAM12 was found with secreted protein acidic and cysteine rich (SPARC), a-smooth muscle actin (ACTA2), and fibroblast activation protein (FAP). No strong inverse correlations with tumor cellularity were found. Gene set enrichment analysis revealed a significant enrichment of extracellular matrix and stromal pathway signatures in patients with high ADAM12 expression (data not shown).

Subclasses of PDAC have been defined at the gene expression level. All current classifications identify a subtype that is characterized by mesenchymal features and increased stromal infiltration [Bijlsma et al., 2017. Nat. Rev. 14:333-42]. We found that ADAM12 expression associated with both the Collisson et al. quasi-mesenchymal [Collisson et al., 2011. Nat. Med. 17:500-3] and the Bailey et al. squamous subtype tumors [Bailey et al., 2016. Nature 531:47-52] (data not shown). Patients clustered with the activated stroma signature from Moffit et al. [Moffitt et al., 2015. Nat. Gen. 47:1168-78.] also showed high expression of ADAM12 (data not shown). These analyses show that the expression of ADAM12 associates with poor-prognosis mesenchymal subgroups of PDAC.

### ADAM12 Expression is Driven by Tumor Cell-Derived TGF-β

Several tumor-derived signals have been identified that shape the stroma by activating the cells that reside in it. For instance, transforming growth factor beta (TGF-β) is a strong activator of cancer-associated fibroblasts (CAFs) and pancreatic stellate cells (PSCs) during cancer progression [Apte et al., 1999. Gut. 44:534-41; Kordes C et al., 2005. Pancreas. 31:156-67]. To functionally confirm this activation mechanism to drive ADAM12 expression, we treated human stellate cells with TGF-6 and other ligands known to be involved in tumor-stroma crosstalk. An upregulation of ADAM12 was only apparent in stellate cells treated with TGF-β (data not shown).

ADAM12 exists as soluble proteins [Kveiborg et al., 2008. Int. J. Biochem. Cell Biol. 40:1685-702]. These forms can be generated by shedding of the cell-bound protein, but in humans a soluble isoform (ADAM12-S) also exists. To determine if soluble ADAM12 is produced by activated stellate cells, PS-1 cells were stimulated using TGF-6 or by coculturing with primary PDAC tumor cells, and ADAM12 was measured by ELISA in the supernatant of these cultures (data not shown). As for the transcript analysis, a strong upregulation of ADAM12 was observed following TGF-6 dependent activation of stellate cells. Coculture of human stellate cells with primary tumor cells led to a significant upregulation of soluble ADAM12 that could be blocked by the TGF-6 pathway inhibitor A83-01, confirming that active TGF-6 ligand is present in these cocultures and able to drive ADAM12 secretion in stromal cells [Tojo et al., 2005. Cancer Sci. 96:791-800].

ADAM12 is Elevated in the Serum of PDAC Patients and Predicts Outcome after Resection

Having established the association of ADAM12 with stromal activation and poor-prognosis molecular subclasses, we proceeded to evaluate ADAM12 as a non-invasive biomarker in PDAC. Patients diagnosed with PDAC before therapeutic intervention showed a significant elevation of serum ADAM12 compared to healthy individuals (FIG. 4). The association of serum ADAM12 levels with clinical parameters was analyzed (data not shown). Patients were dichotomized using serum ADAM12 levels determined by receiver-operator-characteristics (ROC, for live-dead resected patients at time of analysis; 316 pg/ml). No correlations of serum ADAM12 with age, primary tumor size, and disease stage were found (data not shown). High ADAM12 levels in the resected cohort associated with poor survival (HR=2.07, p=0.04), as did high CA19-9 levels and high LNR (data not shown). In a multivariate analysis no significant associations were found (data not shown). The impact of serum ADAM12 on overall survival was analyzed by Kaplan-Meier analysis and log-rank test. We found that whereas serum ADAM12 did not significantly associate with survival in unresectable patients, in resected patients higher ADAM12 levels were strongly associated with shorter survival (data not shown). It thus appears that activated stroma, as revealed by high serum ADAM12 levels, contributes to poor disease outcome when the tumor is at a resectable stage.

ADAM12 Levels Predict Favorable Outcome in Patients Treated with Nab-Paclitaxel

The phase III MPACT trial showed survival benefit of nab-paclitaxel with gemcitabine compared to gemcitabine in metastasized PDAC patients, and is relatively well tolerated. To determine if ADAM12 levels associate with response to nab-paclitaxel, we measured its levels in plasma samples from the MPACT cohort [Von Hoff et al., 2013. N. Engl. J. Med. 369:1691-703; Goldstein et al., 2015. J. Natl. Cancer Inst. 107; Von Hoff et al., 2011. J. Clin. Oncol. 29:4548-54].

Baseline samples were measured and it was observed that the decreased sensitivity of detection in plasma (rather than serum) resulted in a considerable number of samples that had undetectable levels of ADAM12 as defined by 2× standard deviation of the optical density of blanks. Dichotomization of the MPACT cohort by this cutoff resulted in groups with similar size across treatment arms and baseline characteristics (data not shown) but significantly worse survival for patients with detectable ADAM12 (data not shown). Univariate Cox regression revealed a HR of 1.41 (1.10-1.81 95% CI; p=0.0062) for detectable ADAM12 (Supplementary Table S6) in this cohort.

When the trial arms were analyzed separately, ADAM12 levels did not significantly associate with survival in patients that received gemcitabine monotherapy (data not shown). Conversely, in patients that received nab-paclitaxel with gemcitabine, undetectable plasma ADAM12 strongly associated with favorable outcome (data not shown). Patients with undetectable ADAM12 showed a median survival benefit of over 4.0 months from the addition of nab-paclitaxel to gemcitabine, as compared to a benefit of 1.9 months for patients with detectable ADAM12. Baseline ADAM12 levels were significantly associated with outcome in a multivariate model including KPS, and treatment as factors (data not shown). Inclusion of CA19.9 in the model yielded a non-significant association of ADAM12 with survival.

Next, we determined the predictive power of the change in ADAM12 levels during treatment from cycle 1, day 1 (baseline) to cycle 2, day 1 (follow-up) samples (data not shown). A reduction in ADAM12 levels is likely to be caused by a diminished stromal activation, or a reduced tumor load. Indeed, a reduction in plasma ADAM12 associated with improved survival (data not shown). Importantly, these associations were all driven by the favorable outcome of patients with reduced (OS 11.2 m) or repeatedly undetectable (OS 14.4 m) ADAM12 in the nab-paclitaxel with gemcitabine-treated trial arm, as compared to the increased levels (OS 8.3) of ADAM12 (data not shown). Although statistical significance was not reached in the gemcitabine arm, the rank ordering of the groups defined by change in ADAM12 levels on treatment was the same in both arms of the trial. In the poorest-outcome group (in which ADAM12 became detectable or increased at cycle 2), the overall survival was numerically superior at 8.3 months in the nab-paclitaxel with gemcitabine arm as compared to the monotherapy arm at 6.9 months. Thus, our results do not indicate an absence of treatment benefit from nab-paclitaxel in the patients with high or increased ADAM12.

In conclusion, we established that ADAM12 is a serum-borne proxy for the stromal activation of pancreatic cancers, and that its levels associate with poor disease outcome. A low level of or decrease in ADAM12 is associated with improved survival in patients treated with nab-paclitaxel and gemcitabine, and could possibly be used to stratify patients in future trials using this or other treatment combinations.

### Example 2: Stromal Derived Interleukin-6 Drives Epithelial-to-Mesenchymal Transition and Therapy Resistance in Esophageal Adenocarcinoma

Materials and Methods

Informed consent procedure. Signed informed consent was obtained for all patients included in the BiOES biobank according to procedures approved by the Academic Medical Center's ethical committee (MEC 01/288 #08.17.1042). This consent covers all procedures described in this manuscript, including the collection of clinical data, tissue and blood for marker analysis and expansion as xenografts, and in vitro cultures.

Establishment of primary cancer associated fibroblasts. Primary EAC associated fibroblasts were established from resected tumor specimens of EAC patients treated at the Academic Medical Center (Amsterdam, The Netherlands) according to the CROSS regimen (carboplatin, paclitaxel and radiation)[Shapiro et al., 2015. Lancet Oncol 16:1090-1098]. Fresh tumor pieces were washed three times for 5 minutes with PBS containing penicillin (100 units/mL), streptomycin (500 μg/mL), gentamicin (5 μg/ml), cut into small pieces and resuspended in DMEM containing liberase and DNAse for 45 min. Subsequently, cells were resuspended, passed through a 100 μm cell strainer and spun down. Cells were resuspended IMDM medium containing 8% fetal bovine serum (FBS), L-glutamine (2 mM), penicillin (100 units/mL) and streptomycin (500 ug/mL) and plated in a T25 culture flask. After 48 hours, all non-adherent cells were discarded by washing with PBS. Cells were maintained according to standard culture conditions, and upon reaching 80% confluence, cell sorting by negative selection (EPCAM-negative) was performed to obtain a pure fibroblast culture. Antibodies are listed in Table 1. Primary EAC associated fibroblasts exposed to neoadjuvant chemoradiation used are: AMC-EAC-081RF (081RF) and AMC-EAC-243RF (243RF). Treatment naive primary fibroblasts are AMC-EAC-P117BF (117BF) and AMC-EAC-289BF (289BF).

TABLE 1
List of antibodies used.

| | | |
|---|---|---|
| Antibodies for immunofluorescence | Dilution | Clone number, manufacturer |

Primary

| | | |
|---|---|---|
| anti-α-SMA | 1:100 | ab5694, Abcam |
| anti-FLAG | 1:500 | clone M2, Sigma |

Isotype control

| | | |
|---|---|---|
| unconjugated IgG rabbit isotype | 1:300 | DA1E, Cell Signaling |

Secondary

| | | |
|---|---|---|
| Alexa Fluor 488 conjugated anti-rabbit | 1:400 | A11008, Thermo Fisher |
| IgG | | |

| | | |
|---|---|---|
| Antibodies for flow cytometry | Dilution | Clone number, manufacturer |

Primary

| | | |
|---|---|---|
| FITC conjugated anti-HER2 Affibody | 1:1500 | N/A, Bromma, Sweden |
| anti-ERBB3 | 1:1500 | SGP1, Abeam |
| PE conjugated anti-CD24 | 1:50 | ML5, BD Biosciences |
| APC conjugated anti-CD29 | 1:50 | MAR4, BD Biosciences |
| FITC conjugated anti-EPCAM | 1:500 | Ber-EP4, DAKO |
| anti-CXCR4 | 1:100 | UMB2, Abeam |
| APC conjugated anti-CD44 | 1:50 | G44-26, BD Biosciences |
| APC conjugated anti-CD133 | 1:25 | AC133, MACS Miltenyibiotec |
| Biotin conjugated anti-LGR5 | 1:100 | 4D11F8, BD Biosciences |
| anti-Vimentin | 1:100 | 0.N.602, Santa Cruz |

Isotype controls

| | | |
|---|---|---|
| Biotin conjugated LGR5 isotype Rat IgG2b | 1:100 | A95-1, BD Biosciences |
| PE conjugated IgG2a mouse isotype | 1:50 | G155-178, BD Biosciences |
| APC conjugated IgG2b mouse isotype | 1:50 | MPC-11, Biolegend |
| APC conjugated IgG1 mouse isotype | 1:50 | MOPC-21, BD Biosciences |
| FITC conjugated IgG1, K mouse isotype | 1:200 | P3.6.2.8.1, eBioscience |
| unconjugated IgG mouse isotype | 1:100 | X40, BD Biosciences |
| unconjugated IgG rabbit isotype | 1:100 | DA1E, Cell Signaling |

Secondary antibodies/probes

| | | |
|---|---|---|
| APC conjugated anti-mouse | 1:800 | 550826, BD Biosciences |
| Alexa Fluor 488 conjugated anti-rabbit | 1:800 | A-11008, Thermo Fisher |
| IgG | | |
| APC conjugated streptavidin | 1:1000 | 17-4317-82, eBiosience |

| | | |
|---|---|---|
| Reagents for cell sorting | Dilution | Clone number, manufacturer |

Primary antibodies/probes

| | | |
|---|---|---|
| 7-AAD | 1:100 | N/A, BD Biosciences |
| FITC conjugated anti-EPCAM | 1:500 | Ber-EP4, DAKO |
| Anti-EGFR | 1:2000 | H11, Dako |

Secondary

| | | |
|---|---|---|
| APC conjugated anti-mouse | 1:800 | 550826, BD Biosciences |

| | | |
|---|---|---|
| Antibodies for Western blot | Dilution | Clone number, manufacturer |

Primary

| | | |
|---|---|---|
| anti-pSTAT3 (Tyr705) | 1:1000 | D3A7, Cell Signaling |
| anti-STAT3 | 1:1000 | 79D7, Cell Signaling |
| anti-GAPDH | 1:5000 | 6C5, BioConnect |

Secondary

| | | |
|---|---|---|
| HRP-conjugated Goat anti rabbit | 1:5000 | 7074, Cell Signaling |
| HRP-conjugated Goat anti mouse | 1:5000 | 1031-05, Southern Biotech |

Establishment of primary tumor cell cultures. Primary cultures were established as described before from patient-derived xenografts (PDXs) [Damhofer et al., 2015. J Transl Med 13:115]. Briefly, tumor material of patients diagnosed with EAC in the Academic Medical Centre (Amsterdam, The Netherlands) was obtained in accordance with approval by the institute's ethical committee (MEC 01/288 #08.17.1042) [Damhofer et al., 2015. J Transl Med 13: 115]. The tumor material was expanded in NOD.Cg-Prkdc^{scid} Il2rg^{tm1Wjl}SzJ (NSG) immunodeficient mice. Ethical approval was obtained (LEX102774), and the NSG mice were bred and maintained at the local animal facility according to local legislation. Cultures were maintained in Advanced DMEM/F12 (Gibco) with 1:100 N2 (Invitrogen, Carlsbad, Calif.), 2 mM L-glutamine (Sigma-Aldrich, St. Louis, Mo.), 5 mM HEPES (Life Technologies, Carlsbad, Calif.), 0.15% D-glucose (Sigma-Aldrich), 100 μM β-mercaptoethanol (Sigma-Aldrich), 10 μg/ml insulin (Sigma-Aldrich), 2 μg/ml heparin (Sigma-Aldrich), 1:1000 trace elements B and C (Fisher Scientific, Carlsbad, Calif.). The primary EAC cultures used are: AMC-EAC-007B (007B), which was established from a pre-treatment biopsy diagnosis and AMC-EAC-031M (031M), established from a pre-treatment biopsy of a liver metastasis of esophageal adenocarcinoma.

Immunofluorescence was performed as previously described [Ebbing et al., 2017. Gastroenterology 153: 63-76 e14.]. For cell culture and viability assay, flow cytometry, limiting dilution assays, migration assay, quantitative RT-PCR, Survival, gene set enrichment analysis and gene correlations. See Supplementary Materials and Methods.

Cytokine array. The growth factor array AAH-CYT-4000 (RayBiotech, Norcross, Ga.) was performed according to the manufacturer's protocol using three days incubated 081RF supernatant, with unconditioned supernatant as control. Detection was carried out using a FuijFilm LAS4000 and spot intensity was quantified using Image J. Fold induction was calculated according to the manufacturer's instructions; each value was controlled for the positive control spots on each membrane and the values obtained from the unconditioned culture medium. The fold induction values represent the average of duplicate measurements from the membrane. For mouse-derived CAFs, the Mouse Cytokine Array C2000 (RayBiotech) was used. For IL-6 measurements on cell cultures, human IL-6 ELISA DuoSet (R&D systems) was performed on three days conditioned supernatant from OE19, OE33, 007B, 031M, 081RF, 117BF and 268BF cells (all at 80% confluency) and five days conditioned supernatant from 243RF cells (30% confluent). The appropriate unconditioned medium was used as control. Measurements were performed in triplicates and calculated according to a standard curve, according to the manufacturer's protocol. Levels of IL-6 found in CAF supernatants did not exceed the detection range of the ELISA. For mouse CAFs, mouse IL-6 ELISA DuoSet was used. Range for IL-6 ELISA was 1-4000 pg/ml; range for ADAM12 ELISA was 0-18659 pg/ml.

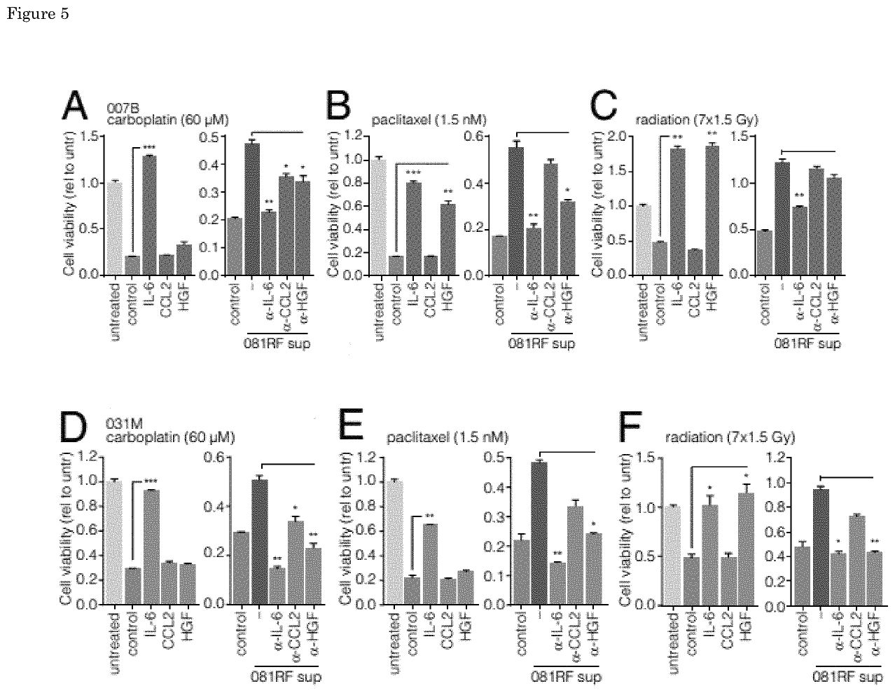

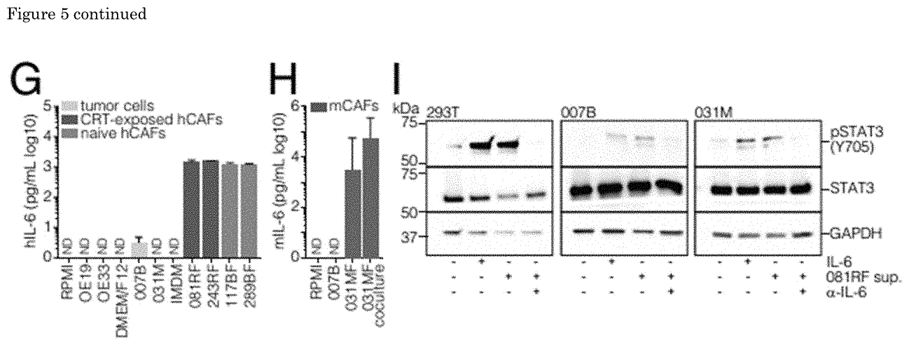

Western blot. Western blots were performed as previously described [Ebbing et al., 2016. Oncotarget 7:10243-10254]. Following transfer, membranes were cut to allow detection of multiple antigens, guided by pre-stained molecular weight markers. Dashed boxes indicate crops shown in FIG. 5I. Primary antibodies were incubated overnight at 4° C. Proteins were imaged using Lumi-Light plus Western blot substrate (Pierce, Thermo Scientific) on a FujiFilm LAS 4000 imager. In parallel to the ECL images, epi-illuminated photographs were captured to document membrane topology.

Organoid cultures. Early passage PDX-derived organoids (P1-10) were cultured in 24-well plates in drops of 50 μl Matrigel (Corning) and maintained in serum free Advanced DMEM/F12 (Gibco), supplemented with N2 supplement (Invitrogen), 2 mM L-glutamine, 100 μM β-mercaptoethanol (Sigma), trace elements B and C (Fisher Scientific), 5 mM HEPES (Life Technologies), 2 μg/ml heparin (Sigma), 10 μg/ml insulin (Sigma), 0.15% D-glucose (Sigma). For passaging, organoids and Matrigel were mechanically disrupted in un-supplemented Advanced DMEM/F12 medium (wash medium). The organoids were washed and resuspended two times prior to passaging. Different culture conditions as indicated were maintained during the assay, chemo-radiation was given prior to passaging. Medium was refreshed twice a week.

Clonogenic assay. 007B and 031M organoids either cultured in control medium, medium containing 25% 081RF supernatant, or 25% 081RF supernatant which was 30 minutes pre-incubated with IL-6 neutralizing antibody were treated with paclitaxel, carboplatin at the indicated concentrations and irradiated (7×1Gy). After two weeks, the organoids were re-plated in 6-well plates at a density of 2000 cells/well. Colony forming was determined after four weeks, using crystal violet. During the assay, the culture medium was refreshed twice a week according to the conditions stated above.

Serum marker analysis. Serum from patients diagnosed with EAC in the Academic Medical Center (Amsterdam, The Netherlands) was collected and stored at −80° C., as approved by the institute's ethical committee (MEC 01/288 #08.17.1042). Informed consent was obtained of all included patients, and blood was drawn prior to start of neoadjuvant treatment according to the CROSS regimen. The human IL-6 and human ADAM12 ELISA (both R&D Systems DuoSet) were performed according to the manufacturer's procedures.

Statistical analysis. For cell viability curves, 2-way ANOVA tests were used to determine statistical significance. For all the other experiments, one-way ANOVA tests were performed, unless noted otherwise. P-values and the R-values of gene expression correlations were determined by linear regression analysis. For the survival analysis, statistical significance was determined using Log-rank (Mantel-Cox) test. For comparison of tumor take in mice, the χ² test was used. All statistical analyses were performed using GraphPad Prism 7. Error bars show the mean±SEM. A P-value of <0.05 was considered statistically significant.

Results

Patient-derived EAC associated fibroblasts confer resistance to chemo- and radiotherapy. To investigate a possible contribution of CAFs to resistance against conventional chemo- and radiation therapy, primary EAC-associated fibroblasts were isolated from resected specimens from patients who received paclitaxel with carboplatin and radiation (the CROSS regimen)[Shapiro et al., 2015. Lancet Oncol 16:1090-1098] (data not shown). Cells were stained with anti-αSMA to confirm their activated myofibroblast-like state (data not shown). Neoadjuvant chemoradiation is standard of care in many Western European countries and the United States. Therefore, CAFs derived from resection specimens will often have been exposed to this treatment. Two previously established EAC cell lines, OE19 and OE33, were treated with carboplatin, paclitaxel, or radiation in the absence or presence of CAF supernatant. CAF supernatant was found to confer resistance against the applied therapeutics (data not shown), as well as other clinically relevant agents such as 5-fluorouracil (5-FU), cisplatin, and eribulin (data not shown). Of note, cells that survived the therapy showed a shift in morphology (data not shown). Tumor cell sensitivity to chemotherapeutics was not influenced by the addition of IMDM (CAF) medium (data not shown).

Using mouse CAFs derived from patient derived xenografts (PDXs), no protective effect was observed (data not shown). These results show that EAC-associated fibroblasts confer resistance by secretion of a molecule that harbors species-specific activity.

To ascertain that the CAF-induced resistance is conserved across different EAC cultures, experiments were performed using the supernatant of EAC CAFs isolated from different patients. All conferred resistance to therapy (data not shown). To determine if the weight of the molecule conferring resistance falls within the range at which most proteins exist, CAF supernatant was filtered using 10- and 100 kDa filters. This revealed that the chemo-protective effect was lost from 10 kDa-filtered supernatant, and that it was retained after 100 kDa filtration (data not shown). Having established that the candidate molecule is likely a protein, a cytokine array was used to identify it. This revealed IL-6, CCL2, and HGF to be the three most abundantly CAF-secreted factors (data not shown). Cytokine analysis of mouse CAF (isolated from PDXs) supernatant revealed high expression of the same cytokines (data not shown).

Stromal CAF-secreted IL-6 drives therapy resistance. To assess the association of the candidate cytokines with patient outcome, we performed survival analysis on the publicly available TCGA gene expression set containing non-pre-treated resected esophageal cancer specimen [Cancer Genome Atlas Research et al., 2017. Nature 541:169-175]. Samples from EAC CGA-EAC) patients were dichotomized by median IL6, CCL2 or HGF expression. A significant association with survival was found for IL6 only (data not shown). To functionally address which cytokine was responsible for the CAF-induced treatment resistance, recombinant IL-6, CCL2, HGF, or CAF supernatant pre-incubated with the pertinent neutralizing antibodies was used in cell viability assays on two primary EAC cultures receiving carboplatin, paclitaxel or radiation (FIG. 5A-F). Of the candidates tested, IL-6 most consistently affected therapy resistance.

Next, we examined if IL-6 was specifically produced by CAFs rather than by tumor cells. Indeed, ELISA on cell supernatants showed that IL-6 secretion was restricted to the CAFs and absent from tumor cell cultures (FIG. 5G). The 081RF CAFs were derived from patients treated with neo-adjuvant chemoradiation therapy. To examine if IL-6 secretion is limited to treated CAFs, we queried public gene expression data from pre-treatment EAC biopsies and healthy tissue [Krause et al., 2016. Carcinogenesis 37:356-365], and found that IL6 was also significantly higher expressed in untreated cancerous tissue compared to normal (data not shown). Gene expression analysis of CAFs isolated from esophageal biopsies revealed these cells to be the likely cellular source of IL-6 in both treated and treatment-naive tissues (data not shown) [Saadi et al., 2010. Proc Natl Acad Sci USA 107:2177-2182]. Next, we isolated treatment naive CAFs from biopsies (117BF, 289BF) and found that these also secreted high amounts of IL-6 (FIG. 5G). High IL-6 levels were also found in mouse CAFs (031MF) supernatant, further supporting the notion that IL-6 production is not unique to fibroblasts exposed to neo-adjuvant chemoradiation (FIG. 5H; cocultures with 031M tumor cells also shown).

To investigate if IL-6 secreted by CAFs can activate its canonical pathway in the cancer cells, 007B and 031M cells were stimulated with CAF supernatant, which resulted in STAT3 phosphorylation. The specificity of this effect was confirmed using IL-6 neutralizing antibody (FIG. 5I). These data suggest that the tumor-promoting properties of the EAC stroma are largely driven by CAF-secreted, biologically active IL-6.

CAF-derived IL-6 induces epithelial-to-mesenchymal transition. From the cell viability experiments, a marked change in morphology in the surviving cells was apparent. To identify the events responsible for this, we performed GSEA on the TCGA-EAC dataset using gene sets for biological programs associated with such phenotypic transitions. Samples were dichotomized by median IL6 expression and a significant association was found for a merged set of two previously published epithelial-to-mesenchymal transition (EMT) signatures, and for a stromal infiltration gene set. Additionally, low IL6 expressing tumors associated with an epithelial signature (data not shown).

To further ascertain EMT as the mechanism responsible for IL-6 induced therapy resistance, primary cells were cultured with CAF supernatant, IL-6, or CAF supernatant pre-incubated with IL-6 neutralizing antibody, and morphology was monitored by microscopy. The induction of a mesenchymal morphology was apparent (data not shown). These morphological changes were also observed using supernatant from treatment naive CAFs (data not shown). Using early passage EAC organoids in the same experimental setup, cells in the IL-6 containing conditions were observed to migrate out of the organoid structures and the Matrigel (data not shown). To characterize and quantify these observations at the molecular level, EMT markers were measured by transcript analysis and increased expression of Zinc finger E-box-binding homeobox 1 (ZEB1), Vimentin (VIM), Snail Family Transcriptional Repressor 2 (SNAI2), and N-cadherin (CDH2) was found in the cultures exposed to IL-6. Epithelial markers E-cadherin (CDH1), and Cytokeratin 19 (KRT19) were decreased (data not shown). These changes were confirmed at the protein level by flow cytometry, which showed increased expression of EMT markers C-X-C chemokine receptor type 4 (CXCR4) and VIM, and decreased expression of epithelial-related genes Human epidermal growth factor receptor 2 (ERBB2), Cluster of differentiation 24 (CD24), Integrin beta-1 (CD29), and Epithelial cell adhesion molecule (EPCAM) (data not shown). We and others have previously found human epidermal growth factor receptor 3 (HER3/ERBB3) to be a marker for epithelial cell identity, and this protein was also downregulated following exposure to IL-6 [Ebbing et al., 2017. Gastroenterology 153:63-76 e14; Biddle et al., 2011. Cancer Res 71: 5317-5326; Padua Alves et al., 2013. Stem Cells 31: 2827-2832]. Analysis of the kinetics of EMT onset in response to CAF supernatant revealed this EMT to take place within several days, a time frame in line with the induction of chemoresistance (data not shown). We take these data to show that IL-6 activates EMT in cancer cells and that this is the mechanism through which resistance against commonly used chemotherapeutics is conferred by the stroma.

IL-6 induced EMT is accompanied by an enhanced migratory- and clonogenic capacity. To study the functional effects of the upregulated EMT markers in addition to the morphological changes, Transwell® migration assays were performed and these showed an enhanced migratory capacity following exposure to IL-6 (data not shown). Furthermore, concomitant with the upregulation of EMT markers, cancer stem cell (CSC) markers CD44/CD44, Prominin-1 (PROM1/CD133), and Leucine Rich Repeat Containing G Protein-Coupled Receptor 5 (LGR5/LGR5) were increased (data not shown). This was accompanied by an increased clonogenicity in limiting dilution assays (data not shown), and implies that stroma-derived IL-6 drives many of the EMT-associated biological programs that are known to contribute to poor outcome in cancer.

To allow an assessment of the contributions of IL-6 signaling to tumor growth in vivo, the problem of species incompatibility between IL-6 and its receptor needed to be addressed. To allow mouse-human transsignaling and human-human autocrine signaling, we generated 031M cells expressing the mouse IL-6 receptor (mIL-6Ra) or human IL-6 ligand (hIL-6; and empty vector), respectively. These cells were injected in immunodeficient mice and tumor outgrowth was only observed from cells expressing mIL6Ra or hIL-6 (data not shown), confirming that IL-6 signaling also contributes to clonogenicity in vivo.

Stroma-derived IL-6 confers resistance to radiochemotherapy in EAC patients. In current clinical practice, patients diagnosed with EAC eligible for curative therapy receive neoadjuvant carboplatin, paclitaxel and fractionated radiation (the CROSS regimen [Shapiro et al., 2015. Lancet Oncol 16: 1090-1098]. To study if CAF-derived IL-6 can confer resistance to such a triple modality regimen, we modeled this treatment by determining the combined doses of the regimen components that allow for a near-complete cell killing, similar to the encouraging but often incomplete responses seen in patients. Primary EAC cells were given one dose of carboplatin and paclitaxel and subsequently received seven radiation doses of 1 Gray (Gy). Colony formation was assessed and efficient outgrowth was observed only in the presence of IL-6 (data not shown). To confirm this response in a model system more representative of human disease, early passage PDX-derived EAC organoids (which underwent clonal selection only during graft expansion which ensured the tumor cell origin of the organoids) were subjected to the same treatment, and the ability to passage the cultures after triple modality treatment was determined (data not shown). Organoid outgrowth following passaging was only observed in cultures exposed to IL-6, and did not occur in the control or IL-6 neutralized conditions.

Having identified the molecule responsible for EMT-associated therapy resistance in EAC cells exposed to triple modality treatments, a logical step would be to measure this cytokine in the serum of patients and correlate it to response, yielding a predictive marker that can predict neoadjuvant treatment outcome. Serum samples from 82 EAC patients before start of neoadjuvant chemo-radiotherapy were analyzed for IL-6 and no significant difference was found between patients grouped by tumor response (Mandard score; data not shown), probably reflecting the association of IL-6 with numerous inflammatory conditions. Instead, we identified ADAM12 by correlative gene expression analyses as a more specific marker for stromal CAFs (data not shown). Its expression associated with poor prognosis (data not shown). ADAM12 levels in the serum are known to correlate with disease stage in lung cancer [Roy et al., 2004. J Biol Chem 279: 51323-51330; Rocks et al., 2006. Br J Cancer 94:724-730; Shao et al., 2014. PLoS One 9:e85936], and its expression is mostly confined to stromal cells in gastrointestinal cancers [Yu et al., 2012. PLoS One 7:e43456]. Measuring serum ADAM12 in these patients revealed a strong correlation to circulating IL-6 (data not shown). Of note, high circulating ADAM12 levels significantly correlated with poor response to chemoradiation (Mandard tumor regression grading score of 3-4) in EAC patients (data not shown). Treatment of CAFs with recombinant IL-6, or blocking IL-6 ligand with antibody did not induce or affect ADAM12 secretion, but addition of recombinant TGF-6 did (data not shown). This confirms that ADAM12 is a feature of highly activated CAFs, and that this activation likely does not result from autocrine IL-6 signaling. Future work will have to validate these findings in other cohorts, and confirm if ADAM12 is indeed an accurate measure of IL-6 producing CAFs in the activated EAC stroma and a predictive marker for currently applicable treatments against EAC.

1. A method for treating a human patient having a cancer characterized by at least one stroma-rich tumor, said human patient having increased disintegrin and metalloproteinase domain-containing protein 12 (ADAM12) levels in a bodily fluid, when compared to ADAM12 levels in a bodily fluid of a human control, the method comprising treating the human patient with a stroma-targeting agent.

2. The method according to claim 1, wherein the human patient has at least one tumor size of more than 20 mm in diameter, a weight loss of more than 5%, a lymph node ratio of more than 0.2, or a combination thereof.

3. The method according to claim 2, wherein the tumor size is determined by:
imaging the tumor by esophagogastroscopy, computed tomography, positron emission tomography, magnetic resonance imaging, or a combination hereof; and
determining a largest dimension of the tumor, wherein a length of said largest dimension is used as a proxy for the tumor size.

4. The method according to claim 2, wherein the lymph node ratio is determined by:
evaluating five or more lymph nodes for the presence or absence of cancer cells; and
calculating the lymph node ratio by dividing an amount of lymph nodes with cancer cells by the total amount of evaluated lymph nodes.

5. The method according to claim 1, wherein the human patient has a carbohydrate antigen 19-9 blood concentration of more than 400 kU/L.

6. The method according to claim 2, wherein the carbohydrate antigen 19-9 blood concentration is determined with an enzyme-linked immunosorbent assay (ELISA).

7. The method according to claim 1, wherein an ADAM12 level in the bodily fluid is higher than 150 μg/mL.

8. The method according to claim 1, wherein an ADAM12 level in a bodily fluid is determined with an enzyme-linked immunosorbent assay (ELISA).

9. The method according to claim 1, wherein the cancer is an esophageal cancer, especially an esophageal adenocarcinoma.

10. The method according to claim 1, wherein the bodily fluid is blood, preferably blood serum.

11. The method according to claim 1, wherein the stroma-targeting agent is a modulator of the renin-angiotensin system, an interleukin 6 (IL-6)-targeting agent, or a combination thereof.

12. The method according to claim 11, wherein the modulator of the renin-angiotensin system is losartan.

13. The method according to claim 11, wherein the IL-6-targeting agent is tocilizumab, siltuximab, olokizumab, elsilimomab, clazakizumab, sirukumab, sarilumab, vobarilizumab, or a combination thereof.

14. The method according to claim 1, wherein the stroma-targeting agent is administered orally, intravenously, subcutaneously, intramuscularly or a combination thereof.

15. (canceled)

16. (canceled)

17. The method according to claim 1, wherein the stroma-targeting agent is administered in a dosage of between 0.1 mg and 2000 mg.

18. The method according to claim 1, wherein the stroma-targeting agent is administered once every two weeks.

19. The method according to claim 1, the method further comprising treating the human patient by chemotherapy, radiation therapy, targeted therapy, immunotherapy, hormone therapy, surgery, or a combination thereof.

20. The method according to claim 1, the method further comprising treating the human patient with trastuzumab, paclitaxel, cisplatin, a fluoropyrimidine such as 5-fluorouracil and capecitabine, oxaliplatin, irinotecan, carboplatin, ramucirumab, folinic acid (leucovorin), a combination of trifluridine with tipiracil, or a combination thereof.

21-28. (canceled)

29. The method according to claim 1, wherein the human patient is firstly administered the stroma-targeting agent, secondly treated with surgery, and ultimately treated with chemotherapy.

30. The method according to claim 1, wherein the human patient is firstly treated with radiotherapy, secondly administered the stroma-targeting agent, thirdly treated with surgery, and ultimately treated with chemotherapy.

31. A pharmaceutical composition comprising a stroma-targeting agent for treating a human patient diagnosed with cancer characterized with at least one stroma-rich tumor, and one or more acceptable excipients.

32. The pharmaceutical composition according to claim 31, wherein the stroma-targeting agent is an interleukin 6 targeting agent.

33. The pharmaceutical composition according to claim 31, wherein the stroma-targeting agent is tocilizumab.

34. The pharmaceutical composition according to claim 31, the pharmaceutical composition further comprising at least one chemotherapeutic agent.

35. A pharmaceutical composition, comprising the pharmaceutical composition according to claim 31 and a pharmaceutical composition comprising at least one chemotherapeutic agent.
